SEQUENCE LISTING (8 sequences)

SEQ ID NO: 1 - DNA, 21 bp, Artificial Sequence
  Synthetic construct
gctcagaacccagatgcaaaa

SEQ ID NO: 2 - DNA, 19 bp, Artificial Sequence
  Synthetic construct
aggaagttccctccaacgc

SEQ ID NO: 3 - DNA, 24 bp, Artificial Sequence
  Synthetic construct
ccatggatgaagggtacttttctg

SEQ ID NO: 4 - DNA, 20 bp, Artificial Sequence
  Synthetic construct
cttttaccaaagcagcagcc

SEQ ID NO: 5 - DNA, 18 bp, Artificial Sequence
  Synthetic construct
tacccacgcagatttatt

SEQ ID NO: 6 - DNA, 18 bp, Artificial Sequence
  Synthetic construct
agagtgagaccacgaaga

SEQ ID NO: 7 - DNA, 18 bp, Artificial Sequence
  Synthetic construct
caccaactgggacgacat

SEQ ID NO: 8 - DNA, 18 bp, Artificial Sequence
  Synthetic construct
acagcctggatagcaacg

(19) United States
(12) Patent Application Publication
(10) Pub. No.: US 2022/0347182 A1
(43) Pub. Date: Nov. 3, 2022

(54) METHODS AND COMPOSITIONS FOR THE TREATMENT OF OSTEOARTHRITIS

(71) Applicant: The Board of Trustees of the Leland Stanford Junior University, Stanford, CA (US)

(72) Inventors: Nidhi Bhutani, Stanford, CA (US); Fiorella Carla Grande, Stanford, CA (US)

(21) Appl. No.: 17/765,086

(22) PCT Filed: Oct. 2, 2020
(86) PCT No.: PCT/US20/54004

§ 371 Date: Mar. 30, 2022

Related U.S. Application Data
(60) Provisional application No. 62/909,547, filed on Oct. 2, 2019.

(51) Int. Cl. A61K 31/522; A61K 31/4439; A61P 19/02

(52) U.S. Cl. CPC A61K 31/522; A61K 31/4439; A61P 19/02

(57) ABSTRACT

Provided herein are methods and compositions for reducing inflammation and/or treating osteoarthritis in a patient in need thereof. The methods include administering to the patient an inhibitor of an inflammation amplifying (Inf-A) population of chondrocytes and an activator of an inflammation dampening (Inf-D) population of chondrocytes. The methods include exposing the osteoarthritic chondrocytes to a composition including an effective amount of an ALK5 inhibitor, a JNK kinase inhibitor, a TNFR II receptor inhibitor, and/or IL1R1 receptor inhibitor and an effective amount of a CD24 activator.

### CROSS-REFERENCED APPLICATION

This application claims priority benefit to U.S. Ser. No. 62/909,547 filed Oct. 2, 2019, and is incorporated herein in its entirety.

### STATEMENT AS TO RIGHTS TO INVENTIONS MADE UNDER FEDERALLY SPONSORED RESEARCH AND DEVELOPMENT

This invention was made with Government support under contract R01 AR070865-01 awarded by the National Institutes of Health. The Government has certain rights in the invention.

### BACKGROUND

Osteoarthritis (OA) is a highly prevalent, age-related disease of the joints, characterized by cartilage degeneration, loss of mobility and chronic pain. Work has been done investigating several aspects of its complex etiology, including the contributions of metabolic, epigenetic, genetic, and cellular factors. However, no disease-modifying drugs exist to treat OA, with the current standard of care being limited to pain-management followed by eventual joint replacement. Recent and ongoing work has highlighted the important interplay between aging, inflammation and loss of regenerative potential in multiple tissues. Although cartilage is a relatively simple tissue, with a single cell type being encapsulated in its secreted extracellular matrix (ECM), the variable degree of degeneration associated with each OA patient suggests that understanding this tissue at a single cell level can provide novel insights into the onset and progression of pathology.

Defining the precise subpopulations that constitute cartilage will also aid strategies for cartilage tissue engineering or for enhancing endogenous cartilage regeneration. Unlike other skeletal tissues, cartilage has a remarkably low regeneration potential. Even injuries sustained in youth remain unrepaired, giving rise to the fibro-cartilaginous tissue that can lead to accelerated OA pathology. Multiple studies have explored the putative cartilage stem or progenitor cells (CPCs) in articular cartilage by characterizing their cell-surface markers and describing their function. Strikingly, the CPC populations were reported to be enriched in OA cartilage, having an increased migratory potential, the ability to form highly clonal populations and multipotency i.e. the ability to give rise to chondrocytes, osteoblasts and adipocytes in culture. Recently, the human skeletal stem cell was identified, further suggesting another fountain of cells for repair. However, despite the existence of these putative regenerative populations, overall cartilage repair remains low, both in healthy and diseased states. Cartilage repair is variable even in younger, non-OA patients who undergo cartilage related injuries, such as anterior cruciate ligament (ACL) rupture or degenerative meniscal tears (DMT), with some patients having a good recovery while others developing OA over a decade or so. Collectively, this suggests that there are factors preventing effective repair and regeneration of the tissue—and that these factors vary between patients.

One source of this limited repair might be the chronic inflammation experienced by the joint. The synovium is known to be infiltrated by a variety of immune cells, and several inflammatory cytokines have been detected in the synovial fluid of OA patients. Further, several studies have characterized the actions of the hypoxia factors (HIF), nitric oxide, reactive-oxygen species, NFK-b signaling, and other pathways that maintain the pro-inflammatory environment. (See for example Refs. 1-7).

There remains a need for effective treatment of osteoarthritis.

### BRIEF SUMMARY OF THE INVENTION

Provided herein are methods of treating osteoarthritis in a subject in need thereof comprising administering an effective amount of an activin-like kinase 5 (Alk5) inhibitor, a c-Jun N-terminal kinase (INK) inhibitor, a tumor necrosis factor receptor II (TNFR II) inhibitor, an interleukin 1 receptor type 1 (IL1R1) inhibitor, or a CD24 activator.

Provided herein are methods of treating osteoarthritis in a patient in need thereof including administering to the patient an inhibitor of an inflammation amplifying (Inf-A) population of chondrocytes and an activator of an inflammation dampening (Inf-D) population of chondrocytes.

Provided herein are compositions including an inhibitor of an inflammation amplifying (Inf-A) population of chondrocytes and an activator of an inflammation dampening (Inf-D) population of chondrocytes.

### BRIEF DESCRIPTION OF THE DRAWINGS

FIGS. 1A and 1B provide high-dimensional profiling of normal and OA chondrocytes using mass cytometry. FIG. 1A shows a schematic outlining the procedures used to profile chondrocytes by mass cytometry. Briefly, cells are dissociated from cartilage tissue, stained with metal conjugated antibodies and analyzed using cyTOF. The resulting data is then gated for live, SOX9/CD44 positive chondrocytes that are used for downstream analyses, including identifying clusters with FlowSOM. FIG. 1B are tSNE plots of 9000 normal chondrocytes, colored by the expression of two chondrogenic markers (SOX9, CD44), the cell surface receptor NOTCH-1 and pNFKB. Expression is set at the max of each channel and is comparable between FIGS. 1E and 1F (top panel); and tSNE plots of 9000 OA chondrocytes, colored by the expression of two chondrogenic markers (SOX9, CD44), the cell surface receptor NOTCH-1 and pNFKB. Expression is set at the max of each channel and is comparable between FIGS. 1E and 1F (bottom panel).

FIGS. 2A-G demonstrate that normal and OA cartilage landscape consists of both abundant and rare subpopulations. FIG. 2A is data showing abundance of each of the 20 clusters called by flowSOM analysis in normal samples. Each point represents a single sample. (n=5) FIG. 2B shows abundance of each of the 20 clusters called by flowSOM analysis in OA samples (n=20). Each point represents a single sample. FIG. 2C is data showing expression of cell surface receptors used for delineating the 20 clusters. Expression is averaged between all cells of a given cluster ID. Gray-scale is z-scaled for each protein between all the clusters. FIG. 2D provides tSNE projections of cells from normal and OA samples that are enriched, similar or depleted in OA compared to normal. Graphs are sampled to 9000 cells when possible. Enrichment, depletion or similarity between the ranked means of normal (n=5) and OA (n=20) cluster abundance was tested using an unpaired, two-tailed Mann-Whitney test with Bonferroni correction (alpha=0.0025). Adjusted p-values for all enriched or depleted clusters are 0.002 (**). FIG. 2E shows coefficient of variation (mean divide by standard deviation) for each cluster in normal or OA samples. FIG. 2F shows Shannon's diversity index (H) calculated for each normal and OA sample (see materials and methods). Theoretical max H value is 2.99. Equality between the means H-values for OA (n=20) and normal (n=5) samples was tested using a two-tailed Mann-Whitney test. p-value=0.001 (***). FIG. 2G provides data showing hierarchical clustering of normal and OA samples by cluster abundances. Abundance is scaled to 1. Samples belonging to the three designated groups are labeled at the bottom.

FIGS. 3A-G demonstrates OA patients are differentially enriched in types of cartilage progenitor cells (CPCs). FIG. 3A provides expression of the 13 CPC markers among the clusters that are enriched for them. Expression is scaled to 1 between all clusters. FIG. 3B are tSNE projections of the Type I (depleted), Type II (similar) and Type III (Enriched) CPCs in OA, where each cluster ID is differentiated by gray-scale. Cells are sampled to 9000 when possible. FIG. 3C shows cell cycle analysis for each cluster. Cell cycle stages were analyzed for each cell individually, and then the proportion of the population in GO and in the cell cycle was calculated for each cluster. The percent in the cell cycle is given to the right of each bar graph. FIG. 3D demonstrates cell signaling and other intracellular and cell surface receptor markers for the CPC clusters. Expression is scaled to 1. FIG. 3E shows cluster abundance for each sample in the OA groups and normal cells. Significant is tested with a multiple-test corrected Welch's T-test. For each group of four bars, from left to right the bars represent data for Group C, Group B, Group A and Normal. FIG. 3F demonstrates the correlation between abundance of each cluster, labeled on each axis. Each point represents an OA patient. The full matrix of correlations between clusters in plotted in S3A. FIG. 3G demonstrates change in cluster abundance for each CPC type after kartogenin treatment compared to DMSO controls, plotted for each patient.

FIGS. 4A-O demonstrates identification of a rare immune recruiting population in OA cartilage. FIG. 4A provides a magnified projection of the clusters 15 and 20 from normal and OA samples. FIG. 4B demonstrates quantification of the abundance of clusters 15 and 20 in normal and OA samples. Significance tested using Welch's t-test. Each point represents a sample. FIG. 4C provides magnified projection of clusters 15 and 20 depicting expression of the two cell surface receptors, TNFRII and IL1R1 and of intracellular HIF2A. Expression is scaled to max value in data set for each protein and are comparable across normal and OA samples. Heatmap below the tSNE depicts quantification of average expression in representative chondrocytes (cluster 5) in comparison to clusters 15 and 20. FIG. 4D provides single-cell RNA sequencing data from Ji et al., reanalyzed. Cells expressing TNFRII and IL1R1 were sorted in-silico, their transcriptome was compared to the rest of the OA cells, and used for GO term and STRING analyses. FIG. 4D is the same as in FIG. 4E, for signaling markers pJNK1/2, pNFKB (FIG. 4F) and pSMAD1/5 (FIG. 4G). FIG. 4H provides fold change in cytokines from human 62-plex Luminex array between DMSO and JNK inhibitor treatment. FIG. 4I provides fold change in cytokines from human 62-plex Luminex array between DMSO and NFKB inhibitor treatment. FIG. 4J provides fold change in cytokines from human 62-plex Luminex array between DMSO and Alk inhibitor treatment. FIG. 4K provides raw MFI values for cytokines that were significantly altered between DMSO and JNK treated samples in at least 5 out of 6 tested OA samples. For each group of two bars, the left bar represents data for DMSO vehicle control and the right bar represents data for INK. Significance was first tested for using ANOVA with multiple corrections for the 62 comparisons and then t-test with Tukey's correction was applied for each comparison on a patient by patient sample. Each point represents an independent technical treatment and cytokine analyses for the same patient (n=6 OA patients). FIG. 4L and FIG. 4M are the same as in FIG. 4M but with NFK-B and Alk inhibitors, respectively (n=3 OA patients). In FIG. 4L for each group of two bars, the left bar represents data for DMSO vehicle control and the right bar represents data for NFKB. In FIG. 4M, for each group of two bars, the left bar represents data for DMSO and the right bar represents data for Alk.

FIGS. 5A-L provides data showing CD24⁺ subpopulation mitigates inflammation in OA cartilage. FIG. 5A provides abundance of each cluster per sample. Differences between the means were tested using Welch's t-test. Data for cluster 17 is on right and data for cluster 18 is on the left. FIG. 5B provides heatmaps of chondrogenic markers SOX9 and CD44, as well as CD24. Expression is scaled to the highest expressing cell in the group. FIG. 5C provides single-cell RNA sequencing data from Ji et al (ref), reanalyzed. Cells expressing CD24 with a high Col2a1/Col1a1 ratio were sorted in-silico, their transcriptome was compared to the rest of the OA cells, and used for GO term and STRING analyses. FIG. 5D provides hierarchical clustering of OA samples based on clusters 15, 17, 18 and 20. Abundance is scaled to one for each cluster. Groups are labeled along the x-axis. FIG. 5E provides violin plots of abundance of Clusters 17, 18, 15 and 20 in low and high Inf-D groups. Each sample is represented as a point. Data for low Inf-D is on the left and data for high Inf-D is on the right. FIG. 5F demonstrates the correlation between the abundance of Cluster 20 with Clusters 17+18. 95% CI is shown in grey dashed line. Slope of line tested is significantly non-zero. FIG. 5G provides heatmaps of the average expression of each marker in the given cluster. FIG. 5H demonstrates the fold change in cytokines from human 62-plex Luminex array between control and IBMX treatment. FIG. 5I demonstrates the fold change in cytokines from human 62-plex Luminex array between control and a combined IBMX and JNK inhibitor treatment.

FIGS. 6A-E provides additional data. FIG. 6A demonstrates the ratio between the ΔΔ Ct of Col2a1 and Col1a1 as measured by RT-qPCR. FIG. 6B provides RT-qCPR results of MMP3 gene expression normalized to Normal #3. FIG. 6C provides RT-qCPR results of MMP9 gene expression normalized to Normal #3. FIG. 6D provides RT-qCPR results of MMP13 gene expression normalized to Normal #3. FIG. 6E. Example gating of live cells on SOX9 and CD44 expression.

FIG. 7 provides a correlation map between 20 OA samples and 5 normal samples. R value is represented by gray-scale.

FIG. 8 is a graph providing the correlation between abundance of Cluster 7 and 9. Each point represents a single OA patient. Collectively, these points give the R² value between Cluster 7 and 9.

FIG. 9 demonstrates the average expression of all the cells in a given cluster (8, 15 or 20) for each sample. Squares that are white represent that there were no cells to plot for that given sample. Expression is given for IL1R1, TNFRII, pJNK, pNFKB and pSMAD1/5. Gray-scale is normalized to highest value of each marker and is comparable within each heatmap.

FIGS. 10A-F provide additional data. FIG. 10A shows a limited cyTOF panel was used to test the age dependance of CD24 expression. Regions including CD24 positive cells are circled in each sample, and the percent of all cells measured is given. FIG. 10B is data with the same limited panel as in (FIG. 10A), normal chondrocytes were treated with either DMSO or IL1B for 24 hours to stimulate a NFK-B response. FlowSOM analysis was performed on samples before and after treatment. The percent change in pNFKB in each cluster is plotted. CD24 positive cells are in cluster 7. FIG. 10C demonstrates STRING network analysis for CD24 positive cells from the scRNA-sequencing dataset of OA patients. Nodes are related to the immune system and immune signaling or to related to oxidative phosphorylation and mitochondrial homeostasis FIG. 10D provides RT-qPCR data of OA patients treated with IBMX for 48 hours, for CD24. Each point represents an independent replicate of the experiment with cells from a given patient (n=3); significant is tested with Student's t-test. p-value<0.05 (*), 0.001(**). For each group of two bars, the left bar represents data for untreated control. FIG. 10E provides RT-qPCR data of OA patients treated with IBMX for 48 hours, for TFAM, PGC1a, and MMP13. Each point represents an independent replicate of the experiment with cells from a given patient (n=3); significant is tested with Student's t-test. p-value<0.05 (*), 0.001(**), 0.0001(***). For each group of two bars, the left bar represents data for untreated control. FIG. 10F provides RT-qPCR data of OA patients treated with JNK inhibitor, IBMX or the combination IBMX for 48 hours for MMP13. Each point represents an independent replicate of the experiment with cells from a given patient (n=3); significant is tested with Welch's T-test with multiple hypothesis testing (alpha=0.016) to account for multiple drug treatments. No significant differences were found between groups.

FIGS. 11A and 11B show that intra-articular injections of Inf-A inhibitor (JNKII inhibitor) slows down progression of post-traumatic OA in a mouse model. FIG. 11A are representative images of injured or uninjured joint treated with a vehicle control or Inf-A inhibitor (JNK II inhibitor). FIG. 11B are graphs showing summit score and maximum score for assessing damage over the joint in vehicle control or Inf-A inhibitor control treated joints.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### I. Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or sub-value there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, shRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “aptamer” as provided herein refers to oligonucleotides (e.g. short oligonucleotides or deoxyribonucleotides), that bind (e.g. with high affinity and specificity) to proteins, peptides, and small molecules. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for almost any protein target are enriched and identified. Aptamers exhibit many desirable properties for targeted drug delivery, such as ease of selection and synthesis, high binding affinity and specificity, flexible structure, low immunogenicity, and versatile synthetic accessibility.

The term “small molecule” as used herein refers to a low molecular weight organic, inorganic, or organometallic compound. A small molecule may comprise a molecular weight of less than 2000 Daltons. A small molecule may comprise a molecular weight of less than 500 Daltons. A small molecule may comprise a molecular weight of about 50 to 500 Daltons. The term “small molecule” as used herein refers to a low molecular weight organic compound that may regulate a biological process. In embodiments, small molecules are drugs.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and -anomeric sugar-phosphate, backbone modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the TUPAC-TUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or antigen binding domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or antigen binding domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

The term “antigen” as provided herein refers to molecules capable of binding to the antibody binding domain provided herein, wherein the binding site is not the peptide binding site.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

A “therapeutic antibody” as provided herein refers to any antibody or functional fragment thereof (e.g., a nanobody) that is used to treat cancer, autoimmune diseases, transplant rejection, cardiovascular disease or other diseases or conditions such as those described herein.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody.

The phrase “specifically (or selectively) binds to an antibody” or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In embodiments, a sample is a cartilage sample. In embodiments, a sample is a healthy cartilage sample. In embodiments, a sample is an osteoarthritic cartilage sample.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The term “chondrocyte” refers to cells found in healthy cartilage. They produce and maintain the cartilaginous matrix, which consists mainly of collagen and proteoglycans. Although the word chondroblast is commonly used to describe an immature chondrocyte, the term is imprecise, since the progenitor of chondrocytes (which are mesenchymal stem cells) can differentiate into various cell types, including osteoblasts. In embodiments, the chondrocyte is part of an inflammation amplifying (Inf-A) population of chondrocytes, In embodiments, the chondrocyte is part of an inflammation dampening (Inf-D) population of chondrocytes.

Cartilage is a resilient and smooth elastic tissue, a rubber-like padding that covers and protects the ends of long bones at the joints and nerves, and is a structural component of the rib cage, the ear, the nose, the bronchial tubes, the intervertebral discs, and many other body components. It is not as hard and rigid as bone, but it is much stiffer and much less flexible than muscle. The matrix of cartilage is made up of glycosaminoglycans, proteoglycans, collagen fibers and, sometimes, elastin. Cartilage is composed of specialized cells called chondrocytes that produce a large amount of collagenous extracellular matrix, abundant ground substance that is rich in proteoglycan and elastin fibers. Cartilage is classified in three types, elastic cartilage, hyaline cartilage and fibrocartilage, which differ in relative amounts of collagen and proteoglycan. Cartilage does not contain blood vessels (it is avascular) or nerves (it is aneural). Nutrition is supplied to the chondrocytes by diffusion. The compression of the articular cartilage or flexion of the elastic cartilage generates fluid flow, which assists diffusion of nutrients to the chondrocytes. Compared to other connective tissues, cartilage has a very slow turnover of its extracellular matrix and does not repair.

The terms “disease” or “condition” refer to a state of being or health status of a patient or subject capable of being treated with the compounds or methods provided herein. The disease may be an autoimmune disease. The disease may be an inflammatory disease. The disease may be an infectious disease.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

The term “prevent” refers to a decrease in the occurrence of disease symptoms in a patient. As indicated above, the prevention may be complete (no detectable symptoms) or partial, such that fewer symptoms are observed than would likely occur absent treatment.

The term “osteoarthritis” refers to is a degenerative disease that worsens over time, often resulting in chronic pain. The condition occurs when the cartilage that cushions the ends of bones in your joints gradually deteriorates. Cartilage is a firm, slippery tissue that enables nearly frictionless joint motion. Eventually, if the cartilage wears down completely, bone will rub on bone. Osteoarthritis has often been referred to as a “wear and tear” disease. But besides the breakdown of cartilage, osteoarthritis affects the entire joint. It causes changes in the bone and deterioration of the connective tissues that hold the joint together and attach muscle to bone. It also causes inflammation of the joint lining.

The terms “activin receptor-like kinase” and “ALK” refer to proteins that belong to the type I activin receptor family. To date, a number of ALKs have been identified in mammals. These ALKs are transmembrane proteins, known as serine/threonine kinase receptors belonging to the transforming growth factor-β (TGF-β) superfamily. The ALKs harbor a transmembrane domain, an extracellular binding domain and a glycine- and serine-rich sequence (GS) domain. The GS domain is a kinase site activated by the TGF-β superfamily type II receptor and can trigger downstream signal transduction. The ALKs elicit various downstream effects of activin/TGF-β, including cell differentiation, proliferation, apoptosis, migration and adhesion as critical modulators of these biological processes. The term “ALK-5” or “activin receptor like kinase 5” refers to a specific ALK.

The term “SB-431542” refers to a drug candidate developed by GlaxoSmithKline (GSK) as an inhibitor of the activin receptor-like kinase (ALK) receptors, ALK5, ALK4 and ALK7.

The terms “JNK” and “INK kinase” and “c-Jun N-terminal kinases” as used herein refer to proteins originally identified as kinases that bind and phosphorylate c-Jun on Ser-63 and Ser-73 within its transcriptional activation domain. They belong to the mitogen-activated protein kinase family, and are responsive to stress stimuli, such as cytokines, ultraviolet irradiation, heat shock, and osmotic shock. They also play a role in T cell differentiation and the cellular apoptosis pathway.

The JNK signal transduction pathway is activated in response to environmental stress and by the engagement of several classes of cell surface receptors, including cytokine receptors, serpentine receptors, and receptor tyrosine kinases. Whitmarch and Davis, J. Mol. Med. 74: 589 (1996). In mammalian cells, JNK has been implicated in immune response (Su et al., Cell 77: 727 (1994); Rincón et al., Genes Funct. 1: 51 (1997); oncogenic transformation (Xu et al., Oncogene 13: 153 (1996); Raitano et al., Proc. Natl. Acad. Sci. U.S.A. 92: 11746 (1995), adaptive responses to stressful environments (Yang et al., Nature 389: 865 (1997)), maturation and differentiation of immune cells (Dong et al., Science 282: 2092 (1998); Shimokawa et al., Biochem. Biophys. Res. Commun. 251: 374 (1998)) and in the apoptotic response of cells that are targets of the immune system (Xia et al., Science 270: 1326 (1995); Zanke et al., Curr. Biol. 6: 606 (1996); Verheij et al., Nature 380: 75 (1996); and Chen et al., J. Biol. Chem. 271: 631 (1996)).

The terms “tumor necrosis factor receptor superfamily” and “TNFRSF” refers to a protein superfamily of cytokine receptors characterized by the ability to bind tumor necrosis factors (TNFs) via an extracellular cysteine-rich domain. With the exception of nerve growth factor (NGF), all TNFs are homologous to the archetypal TNF-alpha. In their active form, the majority of TNF receptors form trimeric complexes in the plasma membrane. Accordingly, most TNF receptors contain transmembrane domains (TMDs), although some can be cleaved into soluble forms (e.g. TNFR1), and some lack a TMD entirely (e.g. DcR3). In addition, most TNF receptors require specific adaptor protein such as TRADD, TRAF, RIP and FADD or downstream signalling. TNT receptors are primarily involved in apoptosis and inflammation, but they can also take part in other signal transduction pathways, such as proliferation, survival, and differentiation. TNF receptors are expressed in a wide variety of tissues in mammals, especially in leukocytes.

The terms “tumor necrosis factor receptor 2”, “TNFR2”, “tumor necrosis factor receptor superfamily member 1B”, “TNFRSF1B”, and “CD120b” refer to a membrane receptor that binds tumor necrosis factor-alpha (TNFα). TNFR2 is one of two receptors of the cytokines, TNF and lymphotoxin-α.

IL1R1 receptor refers to the receptor that binds Interleukin 1 receptor, type I (IL1R1) also known as CD121a (Cluster of Differentiation 121a), which is an interleukin receptor. This protein is a receptor for interleukin 1 alpha (IL1A), interleukin 1 beta (IL1B), and interleukin 1 receptor antagonist (IL1RA). It is an important mediator involved in many cytokine induced immune and inflammatory responses.

CD24 Signal transducer CD24 also known as cluster of differentiation 24 or heat stable antigen CD24 (HSA) is a protein that in humans is encoded by the CD24 gene. CD24 is a cell adhesion molecule. CD24 is a sialoglycoprotein expressed at the surface of most B lymphocytes and differentiating neuroblasts. It is also expressed on neutrophils and neutrophil precursors from the myelocyte stage onwards. The encoded protein is anchored via a glycosyl phosphatidylinositol (GPI) link to the cell surface. The protein also contributes to a wide range of downstream signaling networks and is crucial for neural development.

“Patient,” “subject,” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein the term “effective amount” is an amount sufficient for a compound to accomplish a stated purpose relative to the absence of the compound (e.g. achieve the effect for which it is administered, treat a disease, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques.

As is well known in the art, therapeutically effective amounts for use in humans can also be determined from animal models. For example, a dose for humans can be formulated to achieve a dose that has been found to be effective in animals. The dosage in humans can be adjusted by monitoring effectiveness and adjusting the dosage upwards or downwards, as described herein. Adjusting the dose to achieve maximal efficacy in humans based on the methods described herein and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the composition being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the composition. Thereafter, the dosage is increased by small increments until the optimum effect under circumstances is reached. Dosage amounts and intervals can be adjusted individually to provide levels of the administered composition effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intra-cerebro-ventricular, intrapleural, intra-parencymal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, etc. Administration also includes direct administration, e.g., directly to a site of inflammation. Direct administration may be via guided delivery, e.g., magnetic resonance imaging (MRI)-guided delivery. In embodiments, the administering does not include administration of any active agent other than the recited active agent.

“Co-administer” is meant that a composition described herein is administered at the same time, just prior to, or just after the administration of one or more additional therapies. The compositions provided herein can be administered alone or can be co-administered to the patient. Co-administration is meant to include simultaneous or sequential administration of the compositions individually or in combination (more than one composition). Thus, the preparations can also be combined, when desired, with other active substances.

The terms “immune response” and the like refer, in the usual and customary sense, to a response by an organism that protects against disease. The response can be mounted by the innate immune system or by the adaptive immune system, as well known in the art.

“Pharmaceutically acceptable excipient” and “pharmaceutically acceptable carrier” refer to a substance that aids the administration of an active agent to and/or absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

An “inhibitor” refers to a compound (e.g. compounds described herein) that reduces activity when compared to a control, such as absence of the compound or a compound with known inactivity.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

### Methods of Use

Provided herein are methods of reducing inflammation in a subject in need thereof. The method includes administering an effective amount of an activin-like kinase 5 (Alk5) inhibitor, a c-Jun N-terminal kinase (INK) inhibitor, a tumor necrosis factor receptor II (TNFR II) inhibitor, an interleukin 1 receptor type 1 (IL1R1) inhibitor, or a CD24 activator. In embodiments, the method includes administering an effective amount of an Alk5 inhibitor. In embodiments, the method includes administering an effective amount of a JNK. In embodiments, the method includes administering an effective amount of a TNFR II inhibitor. In embodiments, the method includes administering an effective amount of an IL1R1 inhibitor. In embodiments, the method includes administering an effective amount of a CD24 activator.

In embodiments, the Alk5 inhibitor is an antibody, a nucleic acid, or a small molecule. In embodiments, the Alk5 inhibitor is an antibody. In embodiments, the Alk5 inhibitor is a nucleic acid. In embodiments, the Alk5 inhibitor is a small molecule. In embodiments, the Alk5 inhibitor is SB431542.

In embodiments, the Alk5 inhibitor is SB431542, Galunisertib, A 83-01, A 77-01, SB 505124, R 268712, IN 1130, SM 16, AZ 12799734, or LY 364947. In embodiments, the Alk5 inhibitor is SB431542. In embodiments, the Alk5 inhibitor is Galunisertib. In embodiments, the Alk5 inhibitor is A 83-01. In embodiments, the Alk5 inhibitor is A 77-01. In embodiments, the Alk5 inhibitor is SB 505124. In embodiments, the Alk5 inhibitor is R 268712. In embodiments, the Alk5 inhibitor is IN 1130. In embodiments, the Alk5 inhibitor is SM 16. In embodiments, the Alk5 inhibitor is AZ 12799734. In embodiments, the Alk5 inhibitor is LY 364947.

In embodiments, the JNK inhibitor is an antibody, a nucleic acid, or a small molecule. In embodiments, the JNK inhibitor is an antibody. In embodiments, the JNK inhibitor is a nucleic acid. In embodiments, the JNK inhibitor a small molecule.

In embodiments, the JNK inhibitor is SP600125, TCS JNK6o, SU 3327, CEP 1347, c-JUN peptide, AEG 3481, TCS JNK 5a, BI 78D3, IQ3, SR 3576, IQ iS, JIP-1, or CC401 dihydrochloride. In embodiments, the JNK inhibitor is SP600125. In embodiments, the JNK inhibitor is TCS JNK6o. In embodiments, the JNK inhibitor is SU 3327. In embodiments, the JNK inhibitor is CEP 1347. In embodiments, the JNK inhibitor is c-JUN peptide. In embodiments, the JNK inhibitor is AEG 3481. In embodiments, the JNK inhibitor is TCS JNK 5a. In embodiments, the JNK inhibitor is BI 78D3. In embodiments, the JNK inhibitor is IQ3. In embodiments, the JNK inhibitor is SR 3576. In embodiments, the JNK inhibitor is IQ 1S. In embodiments, the JNK inhibitor is JIP-1. In embodiments, the JNK inhibitor is CC401 dihydrochloride.

In embodiments, the JNK kinase inhibitor is a JNKI kinase inhibitor. In embodiments, the JNK kinase inhibitor is selected from a JNK1 inhibitor and a JNK2 inhibitor. In embodiments, the JNK kinase inhibitor is a JNK1 inhibitor. In embodiments, the JNK kinase inhibitor is a JNK2 inhibitor. In embodiments, the JNK kinase inhibitor is an antibody, nucleic acid, or a small molecule that inhibits JNK kinase activity.

In embodiments, the TNFR II receptor inhibitor is an antibody, nucleic acid, or a small molecule that inhibits the TNFR II receptor or inhibits TNFR II receptor activity.

In embodiments, the TNFR II inhibitor is an antibody, a nucleic acid, or a small molecule. In embodiments, the IL1R1 receptor inhibitor is an antibody, nucleic acid, or a small molecule that inhibits IL1R1 activity. In embodiments, the TNFR II inhibitor is an antibody. In embodiments, the TNFR II inhibitor is a nucleic acid. In embodiments, the TNFR II inhibitor is a small molecule.

In embodiments, the IL1R1 receptor inhibitor is an antibody, a nucleic acid, or a small molecule. In embodiments, the IL1R1 receptor inhibitor is an antibody. In embodiments, the IL1R1 receptor inhibitor is a nucleic acid. In embodiments, the IL1R1 receptor inhibitor is a small molecule.

In embodiments, the CD24 activator is an antibody, nucleic acid, or a small molecule. In embodiments, the CD24 activator is an antibody, nucleic acid, or a small molecule that activates CD24 or increases the activity of CD24 or inhibits an agent that suppresses CD24. In embodiments, the CD24 activator is an antibody. In embodiments, the CD24 activator is a nucleic acid. In embodiments, the CD24 activator is a small molecule. In embodiments, the CD24 activator is 3-isobutyl-1-methylxanthine, also referred to as IBMX.

In embodiments, the method includes administering an effective amount of an activin-like kinase 5 (Alk5) inhibitor and a CD24 activator. In embodiments, the method includes administering an effective amount of a c-Jun N-terminal kinase (INK) inhibitor and a CD24 activator.

In embodiments, the method includes administering an effective amount of a tumor necrosis factor receptor II (TNFR II) inhibitor. In embodiments, the method includes administering an effective amount of an interleukin 1 receptor type 1 (IL1R1) inhibitor.

In embodiments, the method includes administering an effective amount of tumor necrosis factor receptor II (TNFR II) inhibitor and a CD24 activator. In embodiments, the method includes administering an effective amount of an interleukin 1 receptor type 1 (IL1R1) inhibitor and a CD24 activator.

For the methods provided herein, in embodiments, the method includes further administering a pain medication. In embodiments, the pain medication is selected from a non-steroidal anti-inflammatory drug (NSAID), a corticosteroid, a hyaluronic acid, and an opioid. In embodiments, the pain medication is an NSAID. In embodiments, the pain medication is a corticosteroid. In embodiments, the pain medication is a hyaluronic acid. In embodiments, the pain medication is an opioid.

In embodiments, said administering is intraarticularly administering. In embodiments, administering an effective amount of an activin-like kinase 5 (Alk5) inhibitor, a c-Jun N-terminal kinase (INK) inhibitor, a tumor necrosis factor receptor II (TNFR II) inhibitor, an interleukin 1 receptor type 1 (IL1R1) inhibitor, or a CD24 activator is prior to administering of a pain medication. In embodiments, administering an effective amount of an activin-like kinase 5 (Alk5) inhibitor, a c-Jun N-terminal kinase (INK) inhibitor, a tumor necrosis factor receptor II (TNFR II) inhibitor, an interleukin 1 receptor type 1 (IL1R1) inhibitor, or a CD24 activator is at the same times as administering pain medication. In embodiments, administering an effective amount of an activin-like kinase 5 (Alk5) inhibitor, a c-Jun N-terminal kinase (INK) inhibitor, a tumor necrosis factor receptor II (TNFR II) inhibitor, an interleukin 1 receptor type 1 (IL1R1) inhibitor, or a CD24 activator is subsequent to administering pain medication.

In embodiments, the subject is determined to have osteoarthritis by one or more of a physical examination, an x-ray examination, arthroscopic examination, a magnetic resonance examination, and arthrocentesis. In embodiments, the subject is determined to have osteoarthritis by a physical examination. In embodiments, the subject is determined to have osteoarthritis by an x-ray examination. In embodiments, the subject is determined to have osteoarthritis by arthroscopic examination. In embodiments, the subject is determined to have osteoarthritis by a magnetic resonance examination. In embodiments, the subject is determined to have osteoarthritis by arthrocentesis.

For the methods provided herein, in embodiments, treating is reducing the progression of osteoarthritis.

Provided herein are methods of treating osteoarthritis in a patient in need thereof. The method includes administering to the patient an inhibitor of an inflammation amplifying (Inf-A) population of chondrocytes and an activator of an inflammation dampening (Inf-D) population of chondrocytes.

In embodiments, the inhibitor of an inflammation amplifying (Inf-A) population of chondrocytes is an antibody, nucleic acid, or a small molecule. In embodiments, the inhibitor of an inflammation amplifying (Inf-A) population of chondrocytes is selected from a Alk5 inhibitor, INK kinase inhibitor, a TNFR II receptor inhibitor, and IL1R1 receptor inhibitor. In embodiments, the JNK kinase inhibitor is a JNKI kinase inhibitor. In embodiments, the JNK kinase inhibitor is a JNK II inhibitor. In embodiments, the JNK kinase inhibitor is an antibody, nucleic acid, or a small molecule that inhibits JNK kinase activity. In embodiments, the TNFR II receptor inhibitor is an antibody, nucleic acid, or a small molecule that inhibits the TNFR II receptor or inhibits TNFR II receptor activity. In embodiments, the IL1R1 receptor inhibitor is an antibody, nucleic acid, or a small molecule that inhibits IL1R1 activity.

In embodiments, the inhibitor of an inflammation amplifying (Inf-A) population of chondrocytes is a Alk5 inhibitor selected is SB431542. In embodiments, the inhibitor of an inflammation amplifying (Inf-A) population of chondrocytes is a Alk5 inhibitor selected is Galunisertib. In embodiments, the inhibitor of an inflammation amplifying (Inf-A) population of chondrocytes is a Alk5 inhibitor selected is A 83-01. In embodiments, the inhibitor of an inflammation amplifying (Inf-A) population of chondrocytes is a Alk5 inhibitor selected is A 77-01. In embodiments, the inhibitor of an inflammation amplifying (Inf-A) population of chondrocytes is a Alk5 inhibitor selected is SB 505124. In embodiments, the inhibitor of an inflammation amplifying (Inf-A) population of chondrocytes is a Alk5 inhibitor selected is R 268712. In embodiments, the inhibitor of an inflammation amplifying (Inf-A) population of chondrocytes is a Alk5 inhibitor selected is IN 1130. In embodiments, the inhibitor of an inflammation amplifying (Inf-A) population of chondrocytes is a Alk5 inhibitor selected is SM 16. In embodiments, the inhibitor of an inflammation amplifying (Inf-A) population of chondrocytes is a Alk5 inhibitor selected is AZ 12799734. In embodiments, the inhibitor of an inflammation amplifying (Inf-A) population of chondrocytes is a Alk5 inhibitor selected is LY 364947.

In embodiments, the inhibitor of an inflammation amplifying (Inf-A) population of chondrocytes is a JNK kinase inhibitor. In embodiments, the JNK inhibitor is SP600125, TCS JNK6o, SU 3327, CEP 1347, c-JUN peptide, AEG 3481, TCS JNK 5a, BI 78D3, IQ3, SR 3576, IQ 1S, JIP-1, or CC401 dihydrochloride. In embodiments, the inhibitor of an inflammation amplifying (Inf-A) population of chondrocytes is SP600125. In embodiments, the inhibitor of an inflammation amplifying (Inf-A) population of chondrocytes is TCS JNK6o. In embodiments, the JNK inhibitor is SU 3327. In embodiments, the JNK inhibitor is CEP 1347. In embodiments, the inhibitor of an inflammation amplifying (Inf-A) population of chondrocytes is c-JUN peptide. In embodiments, the inhibitor of an inflammation amplifying (Inf-A) population of chondrocytes is AEG 3481. In embodiments, the inhibitor of an inflammation amplifying (Inf-A) population of chondrocytes is TCS JNK 5a. In embodiments, the inhibitor of an inflammation amplifying (Inf-A) population of chondrocytes is BI 78D3. In embodiments, the inhibitor of an inflammation amplifying (Inf-A) population of chondrocytes is IQ3. In embodiments, the inhibitor of an inflammation amplifying (Inf-A) population of chondrocytes is SR 3576. In embodiments, the inhibitor of an inflammation amplifying (Inf-A) population of chondrocytes is IQ 1S. In embodiments, the inhibitor of an inflammation amplifying (Inf-A) population of chondrocytes is JIP-1. In embodiments, the inhibitor of an inflammation amplifying (Inf-A) population of chondrocytes is CC401 dihydrochloride.

In embodiments, the inhibitor of an inflammation amplifying (Inf-A) population of chondrocytes is a TNFR II receptor inhibitor In embodiments, the TNFR II inhibitor is an antibody, a nucleic acid, or a small molecule. In embodiments, the IL1R1 receptor inhibitor is an antibody, nucleic acid, or a small molecule that inhibits IL1R1 activity. In embodiments, the TNFR II inhibitor is an antibody. In embodiments, the TNFR II inhibitor is a nucleic acid. In embodiments, the TNFR II inhibitor is a small molecule.

In embodiments, the inhibitor of an inflammation amplifying (Inf-A) population of chondrocytes is a IL1R1 receptor inhibitor. In embodiments, the IL1R1 receptor inhibitor is an antibody, a nucleic acid, or a small molecule. In embodiments, the IL1R1 receptor inhibitor is an antibody. In embodiments, the IL1R1 receptor inhibitor is a nucleic acid. In embodiments, the IL1R1 receptor inhibitor is a small molecule.

In embodiments, the activator of an inflammation dampening (Inf-D) population of chondrocytes is a CD24 activator. In embodiments, the CD24 activator is an antibody, nucleic acid, or a small molecule that activates CD24 or increases the activity of CD24 or inhibits an agent that suppresses CD24. In embodiments, the CD24 activator is 3-Isobutyl-1-methylxanthine also referred to as IBMX.

Provided herein are methods of decreasing inflammation in osteoarthritic chondrocytes. The method includes exposing the osteoarthritic chondrocytes to an effective amount of a INK kinase inhibitor, a TNFR II receptor inhibitor, and/or IL1R1 receptor inhibitor and an effective amount of a CD24 activator.

In embodiments, methods of decreasing inflammation in osteoarthritic chondrocytes include exposing the osteoarthritic chondrocytes to an effective amount of a INK kinase inhibitor and an effective amount of a CD24 activator. In embodiments, methods of decreasing inflammation in osteoarthritic chondrocytes include exposing the osteoarthritic chondrocytes to an effective amount of a TNFR II receptor inhibitor and an effective amount of a CD24 activator. In embodiments, methods of decreasing inflammation in osteoarthritic chondrocytes include exposing the osteoarthritic chondrocytes to an effective amount of an IL1R1 receptor inhibitor and an effective amount of a CD24 activator.

In embodiments, the JNK kinase inhibitor is a JNKI kinase inhibitor. In embodiments, the JNK kinase inhibitor is a JNK II inhibitor. In embodiments, the JNK kinase inhibitor is an antibody, nucleic acid, or a small molecule that inhibits JNK kinase activity. In embodiments, the TNFR II receptor inhibitor is an antibody, nucleic acid, or a small molecule that inhibits the TNFR II receptor or inhibits TNFR II receptor activity. In embodiments, the IL1R1 receptor inhibitor is an antibody, nucleic acid, or a small molecule that inhibits IL1R1 activity.

In embodiments, the CD24 activator is an antibody, nucleic acid, or a small molecule that activates CD24 or increases the activity of CD24 or inhibits an agent that suppresses CD24. In embodiments, the CD24 activator is 3-Isobutyl-1-methylxanthine also referred to as IBMX.

### II. Compositions

Provided herein are compositions including an effective amount of JNK kinase inhibitor and an effective amount of a (CD24 activator.

Provided herein are compositions including an inhibitor of an inflammation amplifying (Inf-A) population of chondrocytes and an activator of an inflammation dampening (Inf-D) population of chondrocytes.

In embodiments, the JNK kinase inhibitor is a JNKI kinase inhibitor. In embodiments, the JNK kinase inhibitor is a JNK II inhibitor. In embodiments, the JNK kinase inhibitor is an antibody, nucleic acid, or a small molecule that inhibits JNK kinase activity. In embodiments, the TNFR II receptor inhibitor is an antibody, nucleic acid, or a small molecule that inhibits the TNFR II receptor or inhibits TNFR II receptor activity. In embodiments, the IL1R1 receptor inhibitor is an antibody, nucleic acid, or a small molecule that inhibits IL1R1 activity.

In embodiments, the CD24 activator is an antibody, nucleic acid, or a small molecule that activates CD24 or increases the activity of CD24 or inhibits an agent that suppresses CD24. In embodiments, the CD24 activator is 3-Isobutyl-1-methylxanthine also referred to as IBMX.

TABLE 1
Jnk-2 and Alk5 targeting inhibitors

| | | Disease | Cells affected |
|---|---|---|---|
| IL1R1/TNFRII inhibitor | Antibodies, small | Osteoarthritis | Chondrocytes |
| | molecule | | |
| Pan JNK kinase inhibitor | Antibodies, small | Osteoarthritis | Chondrocytes |
| | molecule | | |
| Alk 5 inhibitor | Antibodies, small | Osteoarthritis | Chondrocytes |
| (Activin like kinase 5 | molecule (SB431542) | | |
| inhibitor) | | | |
| IL1R1/TNFRII inhibitor + | Antibodies, small | Osteoarthritis | Chondrocytes |
| CD24 activator | molecule | | |
| Pan JNK kinase inhibitor + | Antibodies, small | Osteoarthritis | Chondrocytes |
| CD24 activator | molecule | | |
| Alk inhibitor + CD24 | Antibodies, small | Osteoarthritis | Chondrocytes |
| activator | molecule | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### P EMBODIMENTS

P Embodiment 1. A method of reducing inflammation in a patient in need thereof, the method comprising administering to the patient an effective amount of a JNK kinase inhibitor, a TNFR II receptor inhibitor, and/or IL1R1 receptor inhibitor and an effective amount of a CD24 activator.

P Embodiment 2. The method of embodiment 1, wherein the JNK kinase inhibitor is an antibody, nucleic acid, or a small molecule.

P Embodiment 3. The method of embodiment 1, wherein the TNFR II receptor inhibitor is an antibody, nucleic acid, or a small molecule.

P Embodiment 4. The method of embodiment 1, wherein the IL1R1 receptor inhibitor is an antibody, nucleic acid, or a small molecule.

P Embodiment 5. The method of any one of embodiments 1 or 2, wherein the JNK kinase inhibitor is selected from a JNK1 and a JNKII inhibitor.

P Embodiment 6. The method of any one of embodiments 1-5, wherein the CD24 activator is an antibody, nucleic acid, or a small molecule.

P Embodiment 7. The method of embodiment 6, wherein the CD24 activator is 3-Isobutyl-1-methylxanthine (IBMX).

P Embodiment 8. A method of treating osteoarthritis in a patient in need thereof, the method comprising administering to the patient an inhibitor of an inflammation amplifying (Inf-A) population of chondrocytes and an activator of an inflammation dampening (Inf-D) population of chondrocytes.

P Embodiment 9. The method of embodiment 8, inhibitor of an inflammation amplifying (Inf-A) population of chondrocytes is an antibody, nucleic acid, or a small molecule.

P Embodiment 10. The method of embodiment 9, wherein the inhibitor of an inflammation amplifying (Inf-A) population of chondrocytes is selected from a JNK kinase inhibitor, a TNFR II receptor inhibitor, and IL1R1 receptor inhibitor.

P Embodiment 11. The method of any one of embodiments 8-10, wherein the activator of an inflammation dampening (Inf-D) population of chondrocytes is a CD24 activator.

P Embodiment 12. The method of embodiment 11, wherein the CD24 activator is 3-Isobutyl-1-methylxanthine (IBMX).

P Embodiment 13. A method of decreasing inflammation in osteoarthritic chondrocytes, the method comprising exposing the osteoarthritic chondrocytes to an effective amount of an JNK kinase inhibitor, a TNFR II receptor inhibitor, and/or IL1R1 receptor inhibitor and an effective amount of a CD24 activator.

P Embodiment 14. A composition comprising an effective amount of JNK kinase inhibitor and an effective amount of a CD24 activator.

P Embodiment 15. A composition comprising an inhibitor of an inflammation amplifying (Inf-A) population of chondrocytes and an activator of an inflammation dampening (Inf-D) population of chondrocytes.

### EMBODIMENTS

Embodiment 1. A method of treating osteoarthritis in a subject in need thereof comprising administering an effective amount of an activin-like kinase 5 (Alk5) inhibitor, a c-Jun N-terminal kinase (JNK) inhibitor, a tumor necrosis factor receptor II (TNFR II) inhibitor, an interleukin 1 receptor type 1 (IL1R1) inhibitor, or a CD24 activator.

Embodiment 2. The method of embodiment 1, wherein the Alk5 inhibitor is an antibody, a nucleic acid, or a small molecule.

Embodiment 3. The method of embodiment 1 or 2, wherein the Alk5 inhibitor is SB431542.

Embodiment 4. The method of any one of embodiments 1-3, wherein the JNK inhibitor is an antibody, a nucleic acid, or a small molecule.

Embodiment 5. The method of any one of embodiments 1-4, wherein the JNK kinase inhibitor is selected from a JNK1 inhibitor and a JNK2 inhibitor.

Embodiment 6. The method of any one of embodiments 1-5, wherein the TNFR II inhibitor is an antibody, a nucleic acid, or a small molecule.

Embodiment 7. The method of any one of embodiments 1-6, wherein the IL1R1 receptor inhibitor is an antibody, a nucleic acid, or a small molecule.

Embodiment 8. The method of any one of embodiments 1-7, wherein the CD24 activator is an antibody, nucleic acid, or a small molecule.

Embodiment 9. The method of embodiment 8, wherein the CD24 activator is 3-Isobutyl-1-methylxanthine (IBMX).

Embodiment 10. The method of embodiment 1, comprising administering an effective amount of an activin-like kinase 5 (Alk5) inhibitor and a CD24 activator.

Embodiment 11. The method of embodiment 1, comprising administering an effective amount of a c-Jun N-terminal kinase (JNK) inhibitor and a CD24 activator.

Embodiment 12. The method of embodiment 1, comprising administering an effective amount of a tumor necrosis factor receptor II (TNFR II) inhibitor.

Embodiment 13. The method of embodiment 1, comprising administering an effective amount of an interleukin 1 receptor type 1 (IL1R1) inhibitor.

Embodiment 14. The method of embodiment 1, comprising administering an effective amount of tumor necrosis factor receptor II (TNFR II) inhibitor and a CD24 activator.

Embodiment 15. The method of embodiment 1, comprising administering an effective amount of an interleukin 1 receptor type 1 (IL1R1) inhibitor and a CD24 activator.

Embodiment 16. The method of any one of embodiments 1-15, further comprising administering a pain medication.

Embodiment 17. The method of embodiment 16, wherein the pain medication is selected from a non-steroidal anti-inflammatory drug (NSAID), a corticosteroid, a hyaluronic acid, and an opioid.

Embodiment 18. The method of any one of embodiments 1-15, wherein said administering is intraarticularly administering.

Embodiment 19. The method of any one of embodiments 16-18, wherein said administering is subsequent to said administering of said pain medication.

Embodiment 20. The method of any one of embodiments 1-19, wherein the subject is determined to have osteoarthritis by one or more of a physical examination, an x-ray examination, arthroscopic examination, a magnetic resonance examination, and arthrocentesis.

Embodiment 21. The method of any one of embodiments 1-20, wherein said treating is reducing the progression of said osteoarthritis.

### EXAMPLES

### Example 1: Single-Cell Mass Cytometry of Osteoarthritic Cartilage

Aging or injury leads to degradation of the cartilage matrix and the development of osteoarthritis (OA). Due to a paucity of single-cell studies of GA cartilage, little is known about the interpatient variability in its cellular composition and, more importantly, about the cell subpopulations that drive the disease. Experiments herein profiled healthy and OA cartilage samples using mass cytometry to establish a single-cell atlas, revealing distinct chondrocyte progenitor and inflammation modulating subpopulations. These rare populations include an inflammation amplifying (Inf-A) population, marked by IL1R1 and TNFRII, whose inhibition decreased inflammation, and an inflammation dampening (Inf-D) population, marked by CD24, which is resistant to inflammation. A pharmacological strategy was devised targeting Inf-A and Inf-D cells that significantly decreased inflammation in OA chondrocytes. OA patients were stratified in three groups that are distinguished by the relative proportions of inflammatory to regenerative cells, making it possible to devise precision therapeutic approaches.

To understand how the milieu found in joint regions might affect the pro-regenerative populations, such as the CPCs, single-cell mass cytometry (cyTOF) was utilized to map both the pro-regenerative cell populations and inflammatory populations. By simultaneously being able to map cell identity and signaling states, how cells interact and influence each other was observed. Furthermore, these maps provided a new, cell-population based stratification of OA patients, which may aid in targeted OA therapeutics in the future.

High-Dimensional Mass-Cytometry Based Profiling of Normal and OA Cartilage

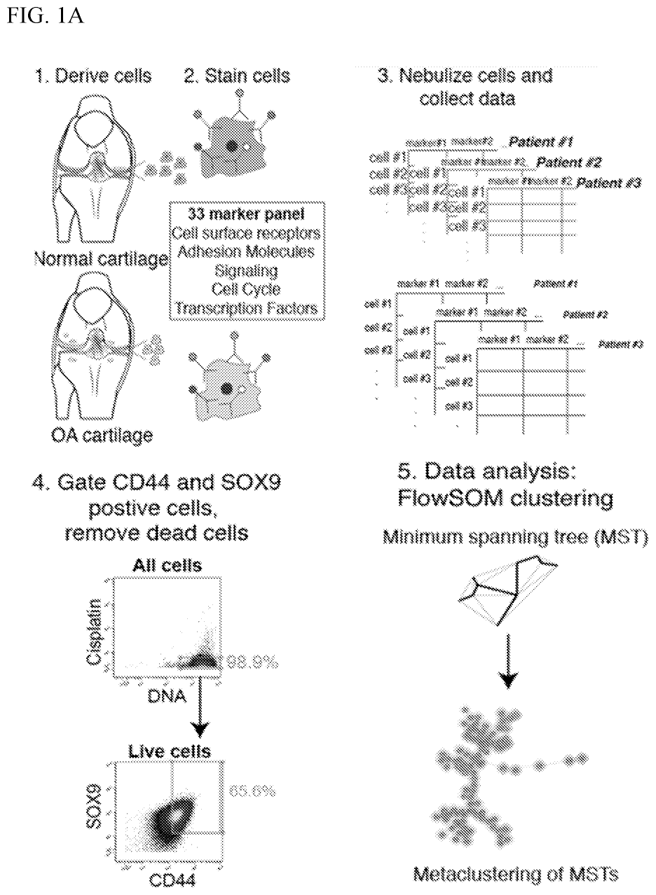

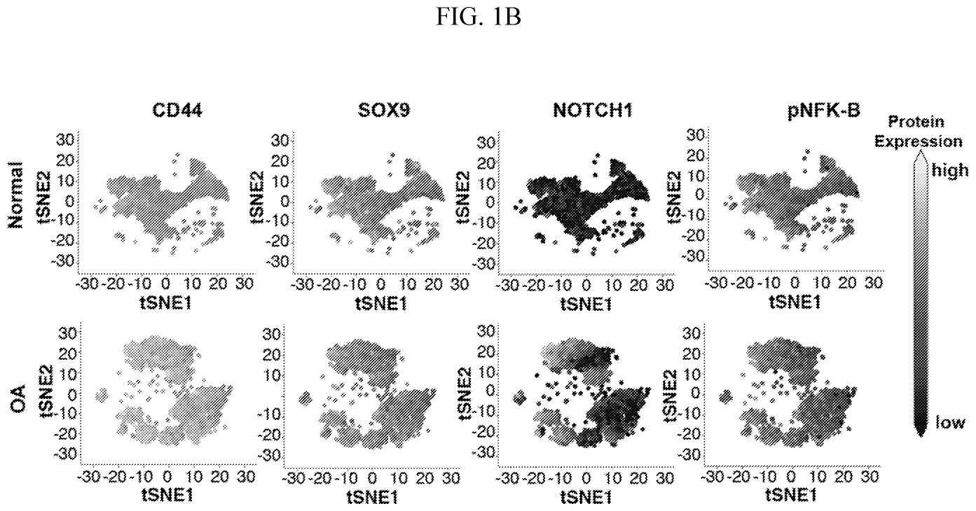

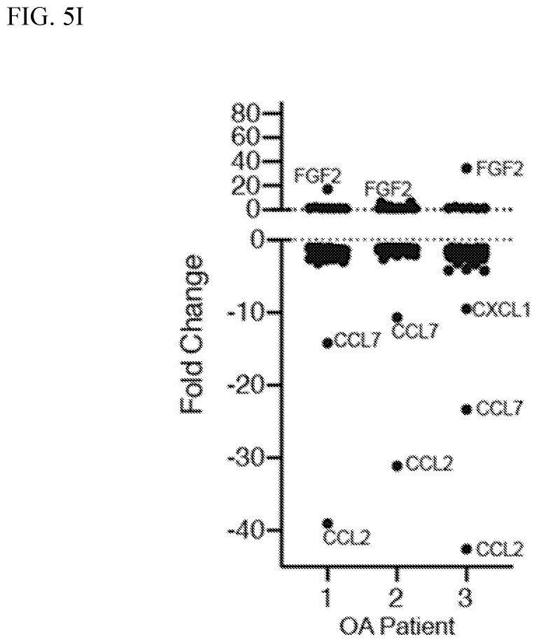

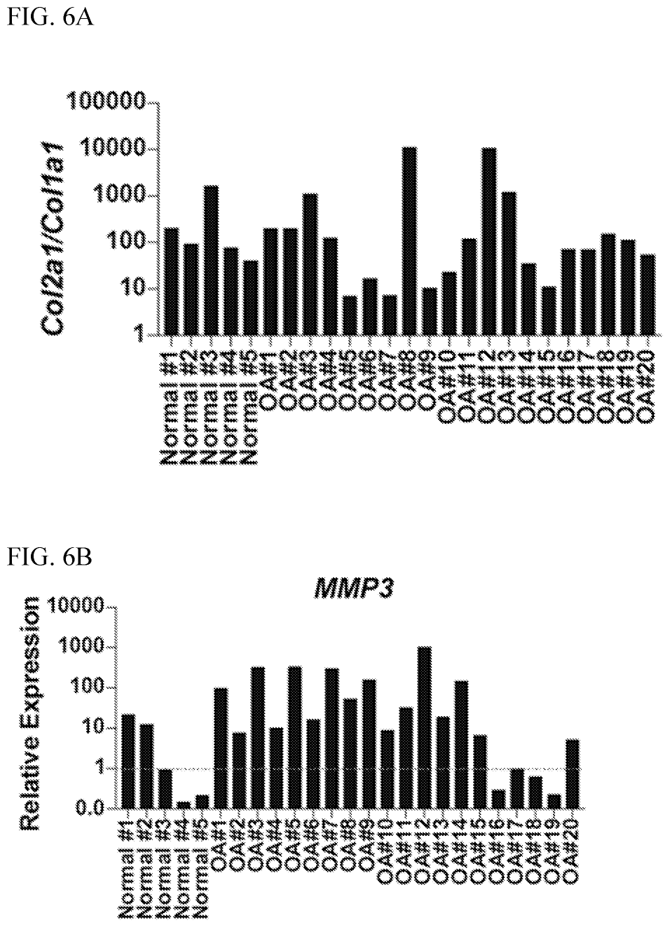

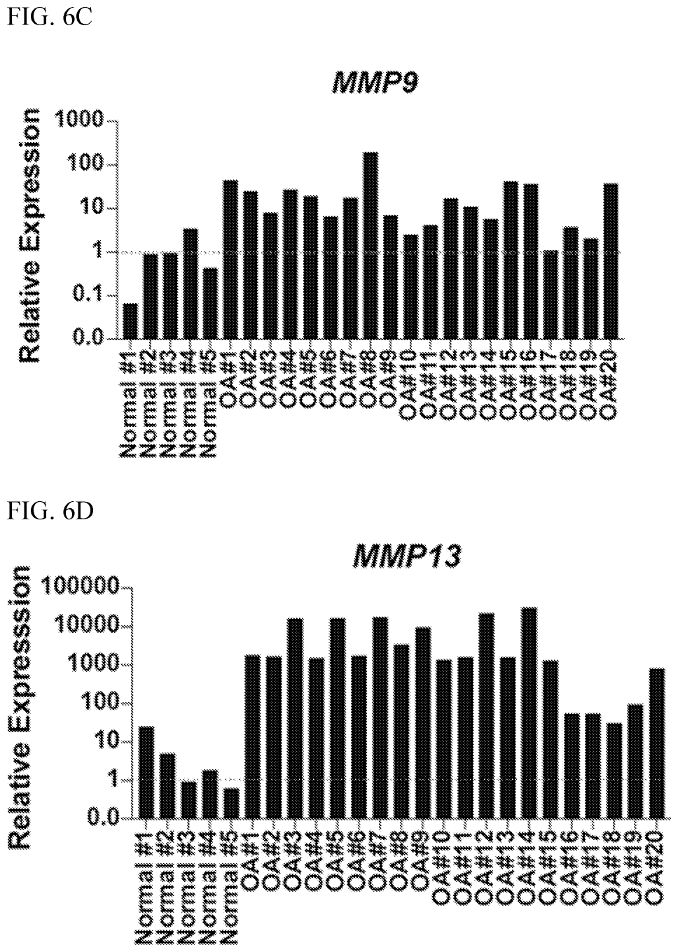

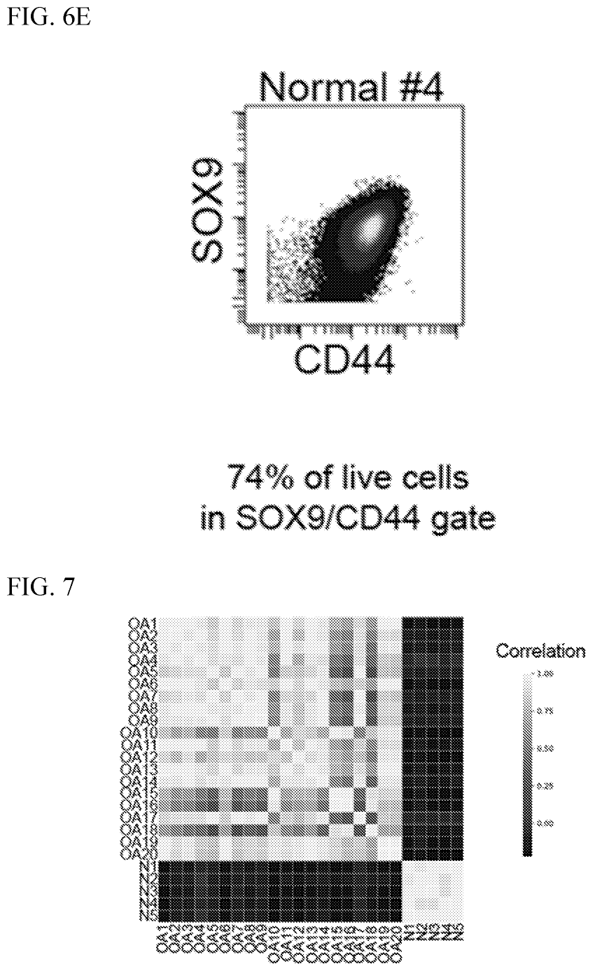

Towards the goal of profiling rare stem/progenitor-like populations within normal and OA cartilage, cytometry by time-of-flight, or cyTOF, a mass-spectrometry based high dimensional method for single-cell detection of isotope labeled antibodies (FIG. 1A) was utilized. While cyTOF panels have to be pre-selected for each experiment, this technique provided the advantage that a large number of cells can be easily profiled in multiple samples without being cost-prohibitive. In addition, this profiling at the protein level was complementary to single cell transcriptomics and can additionally provide a snapshot of the active signaling pathways in a specific subpopulation. After detailed study of the literature and preliminary data, a panel of 33-markers was labeled and optimized (see materials and methods and Table 2) for profiling chondrocytes. This panel included cell surface receptors, adhesion molecules, signaling mediators and cell cycle and transcription factors that are known to be important for cartilage homeostasis (Table 2). Samples were collected from the surgical waste of OA patients undergoing total-knee arthroplasty (according to an IRB protocol approved by Stanford University), digested and expanded for a single passage in high density culture as previously described. Each sample was tested for a high expression ratio of Col2a1/Col1a1 (10-100 fold) (FIGS. 6A-6E) to ensure chondrogenicity and the expression of MMP3, 9 and 13 which is known to be high in OA cartilage (FIGS. 6B-6D). An average of 3×10⁴ and 10×10⁴ cells were assayed per OA or normal sample, respectively and only the Sox9/CD44 double positive cells were further analyzed (FIG. 6E). For visualization, the total population was downsampled to 9%, representing 9000 cells, and cells were projected onto a 2D plane using tSNE. The spatial representation of OA and normal cells was distinct, although no one single sample (patient) for either normal or OA samples was observed to dominate this representation. Analysis of SOX9 and CD44 staining showed high levels of staining across all cells, ensuring the chondrogenic phenotype of the cells with no dedifferentiation observed during sample processing (FIG. 1). The single cell data from 20 OA samples and 5 normal samples were analyzed. Known features of the OA landscape were observed, for example the expansion of NOTCH1 expressing chondrocytes in OA activated Notch positive chondrocyte populations in OA (FIG. 1B). Phosphorylated NFK-B, in contrast, could not readily distinguish between normal and OA samples, which both consisted of populations manifesting high, medium and low level of signaling (FIG. 1B).

TABLE 2
Resources

| | | Staining | | |
|---|---|---|---|---|
| | | concentration | | |
| | | (ug) for | | |
| Antibody | Channel | 2.5*million cells | Clone | Company |
| CD44 | 141 | 0.1 | IM7 | Biolegend |
| Hif2a | 142 | 1 | ep190b | Novus |
| CD121B | 143 | 0.1 | MNC2(Ruo) | BD Biosciences |
| SOD2 | 144 | 3 | D3X8F | Cell Signaling |
| | | | | Technology |
| pStat3(pY705) | 145 | 1 | 4/P-STAT3(Ruo) | BD Biosciences |
| pBcat(Y654) | 146 | 3 | Ab59430 | Abcam |
| Sox9 | 147 | 1 | 3C10 | Abcam |
| pNFKB(S29) | 149 | 1 | K10-895.12.50 | BD Biosciences |
| CD151 | 150 | 2 | 14A2.H1 | BD Biosciences |
| CD73 | 151 | 0.5 | AD2 | BD Biosciences |
| CD166 | 152 | 3 | 3A6 | BD Biosciences |
| CD29 | 153 | 0.5 | TS2116 | Biolegend |
| CD9 | 154 | 0.5 | H19a | Biolegend |
| Notch-1 | 155 | 3 | MHN1-519 | Biolegend |
| CD121A | 156 | 0.1 | H1L1R-M1(Ruo) | BD Biosciences |
| pH3 | 158 | 0.5 | HTA28 | Biolegend |
| CD106 | 159 | 0.5 | 51-10C9 | BD Biosciences |
| CD24 | 160 | 3 | ML5 | Biolegend |
| CD120A | 161 | 0.1 | MABTNFR1-B1 | BD Biosciences |
| pSMAD2(S45/467)/SMAD3 | 162 | 1 | D27F4 | Cell Signaling |
| (S423/425) | | | | Technology |
| pERK1/2(pT202/pY204) | 163 | 3 | 20A | Fisher |
| Stro-1 | 164 | 0.5 | Stro-1 | Biolegend |
| CD120B | 165 | 0.1 | 367A02 | Biolegend |
| pRb(S807/S811) | 166 | 1 | J112-905 | BD Biosciences |
| Cyclin B1 | 167 | 3 | GNS-1 | BD Biosciences |
| CD49E | 169 | 0.1 | VC5 | BD Biosciences |
| pcJun(S73) | 170 | 3 | D47G9) | Cell Signaling |
| | | | | Technology |
| CD49C | 171 | 0.5 | C311.1 | BD Biosciences |
| CD90 | 172 | 0.1 | 5E10 | Biolegend |
| CD105 | 173 | 1 | 43A3 | Biolegend |
| CD54 | 174 | 0.5 | HA58 | BD Biosciences |
| P-SAPK/JNK (T183/Y185) | 175 | 1 | G2 | Cell Signaling |
| | | | | Technology |
| p-SMAD1/5(S463/465) | 176 | 1 | 41D10 | Cell Signaling |
| | | | | Technology |

Normal and GA Cartilage Landscape Consists of Both Abundant and Rare Subpopulations

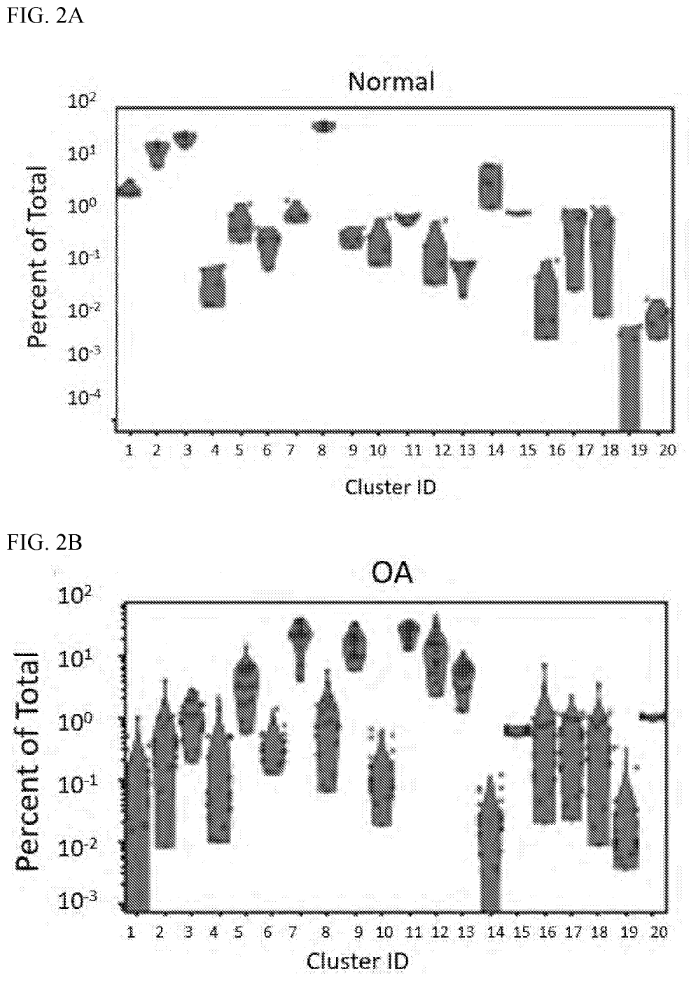

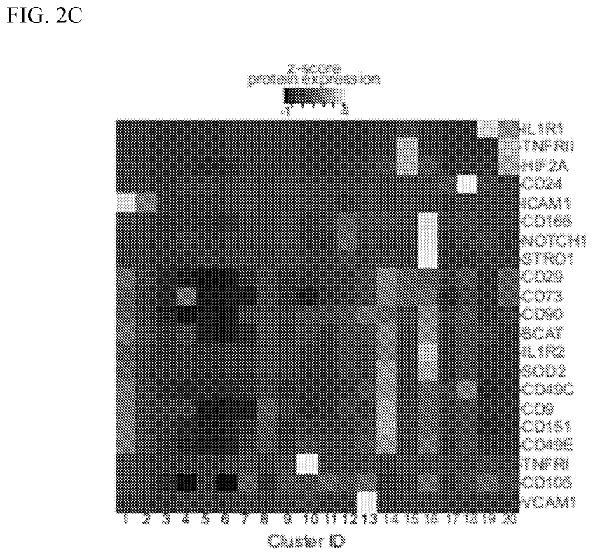

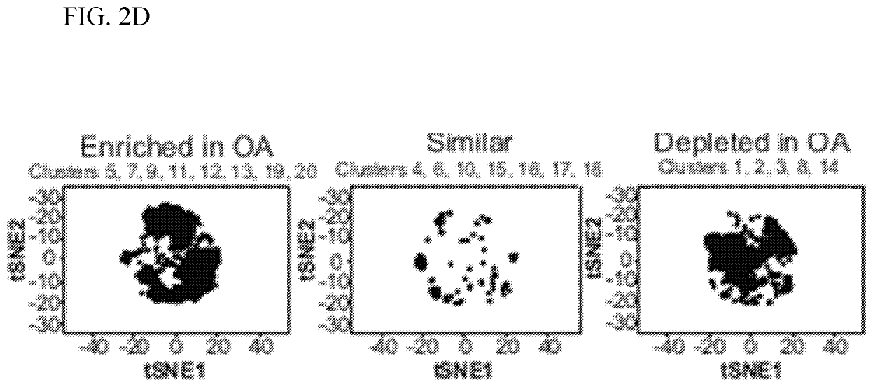

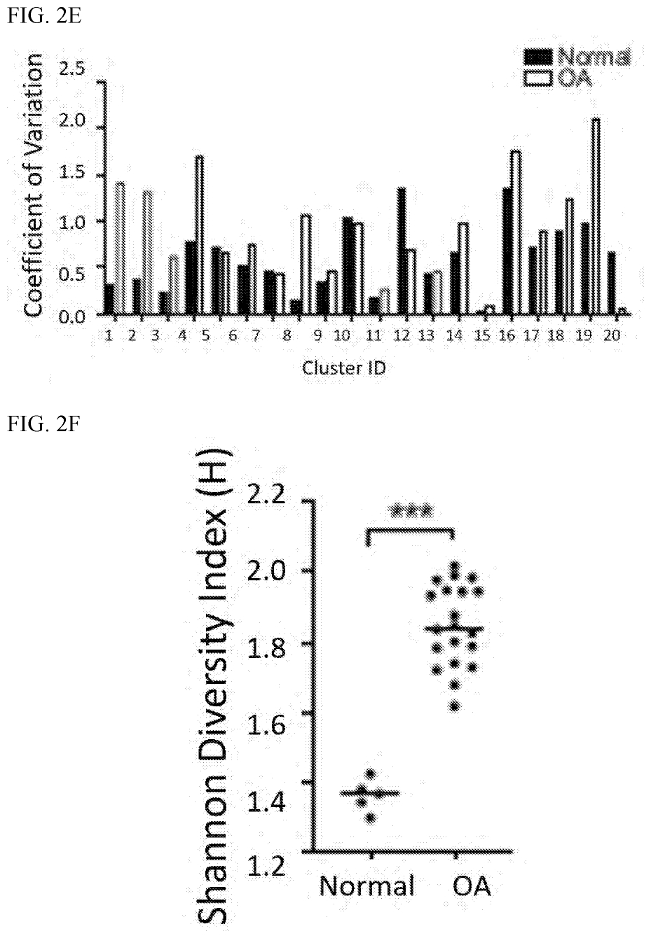

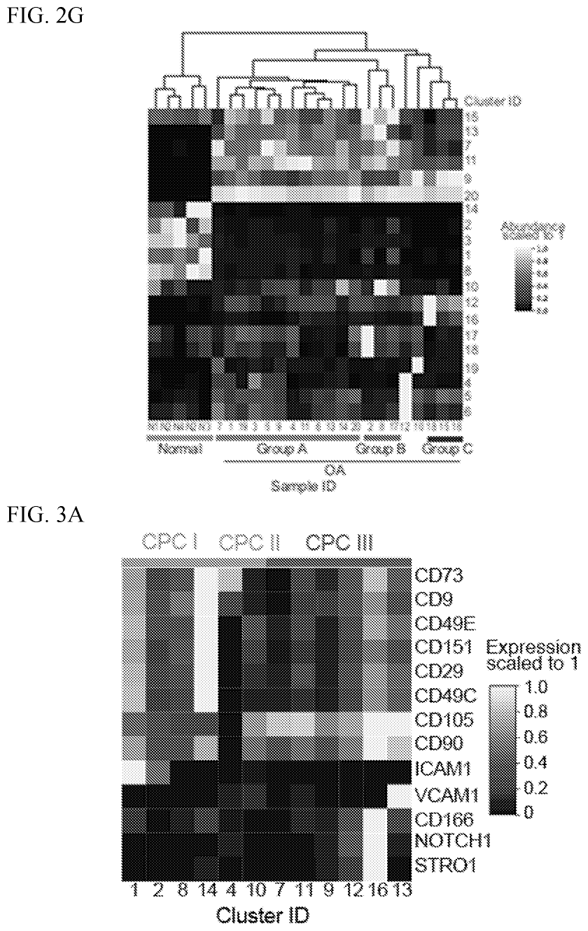

To find unique subpopulations in the normal and GA cartilage, the algorithm FlowSOM was utilized to define clusters (see materials and methods) based on the similarity of expression of cell-surface receptors and intracellular markers (See for example Ref. 10). FlowSOM identified 20 clusters or subpopulations in the data (FIG. 2A). Similar numbers and compositions of clusters were observed using alternate methods of analysis (data not shown). A z-score distribution matrix for all the surface receptors and intracellular markers used to define these clusters (FIG. 2C) demonstrated the molecular identity of these clusters. For example, clusters 1 and 2 were marked by high ICAM, clusters 12 and 16 had a high expression of NOTCH1, STRO1 and CD166 and clusters 10 and 20 had high IL1R1 and TNFRII. Using the 20 clusters, observations showed that the OA patients were highly anticorrelated with the normal samples, validating their sample identity (FIG. 7).

Next, experiments were undertaken to investigate how the nature and frequency of the identified subpopulations varied between the normal and OA samples—specifically to determine if populations were gained or lost during disease progression. Based on this idea, the clusters were categorized into 3 groups: a) increased in OA, b) unchanged between OA and normal, and c) decreased in OA. Eight subpopulations (Cluster 5, 7, 9, 11, 12, 13, 19 and 20) were enriched in the OA samples compared to normal; five subpopulations (Cluster 1, 2, 3, 8 and 14) were depleted compared to normal while seven subpopulations (Clusters 4, 6, 10, 15, 16, 17 and 18) remained unchanged between the OA and normal samples (FIG. 2D). Quantitation of the frequency of these populations revealed inter-patient heterogeneity, which were quantified using the coefficient of variation (FIG. 2E). Across most of the populations, OA samples showed a higher coefficient of variation than their normal counterparts (FIG. 2E), putatively indicating that disease progression alters the homeostasis of subpopulations leading to a greater heterogeneity. As an alternate way to quantify this heterogeneity, a metric used in population ecology was utilized, known as Shannon's Diversity Index, which describes how heterogeneous and evenly distributed populations are in an ecosystem. Based on the 20 populations identified by FlowSOM, data demonstrated that (a) OA samples had a higher Shannon diversity index (H value) and additionally (b), the range of H values for OA patients was much larger than for normal samples indicating a loss of population evenness in OA (FIG. 2F).

Using these populations, whose unique identities are detailed in later sections, hierarchical clustering of the 20 OA patients and 5 normal samples in the study was performed. The goal was to identify subsets of patients with unique compositions of these rare populations. Such characterization, common in the cancer field, can be helpful in designing targeted therapeutic strategies tailored to groups of patients with similar molecular underpinnings driving their disease. As expected, all the normal samples clustered together (FIG. 2G). Data demonstrated 3 major groups of patients, with some patients that clustered only with themselves. Group A, the largest of the three groups with 12 patients was enriched in Clusters 7 and 11, marked by CD105 expression (FIG. 2G). Group B, consisting of three patients, was enriched in Clusters 17 and 18, the CD24 positive populations and Group C, also consisting of three patients, was characterized by a high abundance of clusters 9, 12 and 16, that were identified to be NOTCH1/VCAM-1 positive cartilage progenitor cells (CPC) (FIG. 2G). The following sections will detail the unique characteristics of these populations and the etiology they reveal about the underlying OA patients.

Patients are Differentially Enriched in Inflammatory and Noninflammatory CPCs

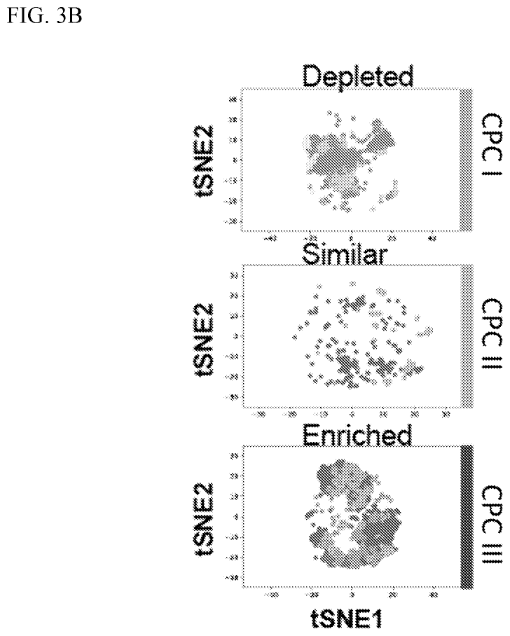

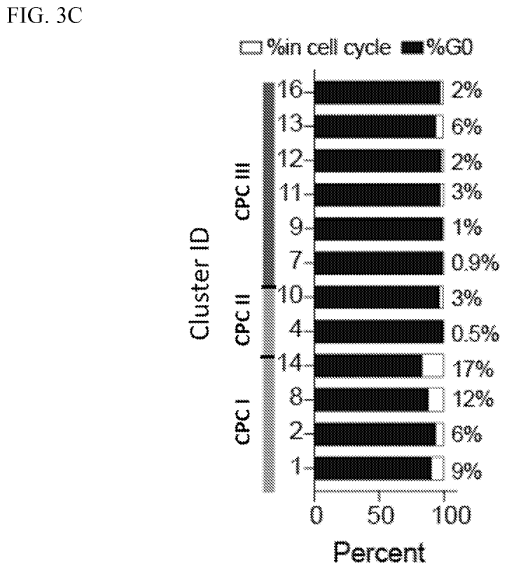

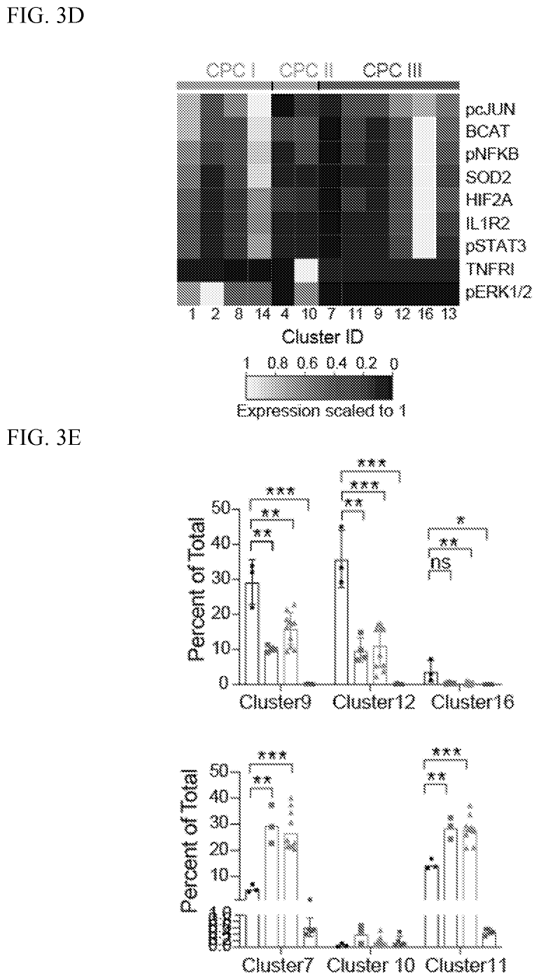

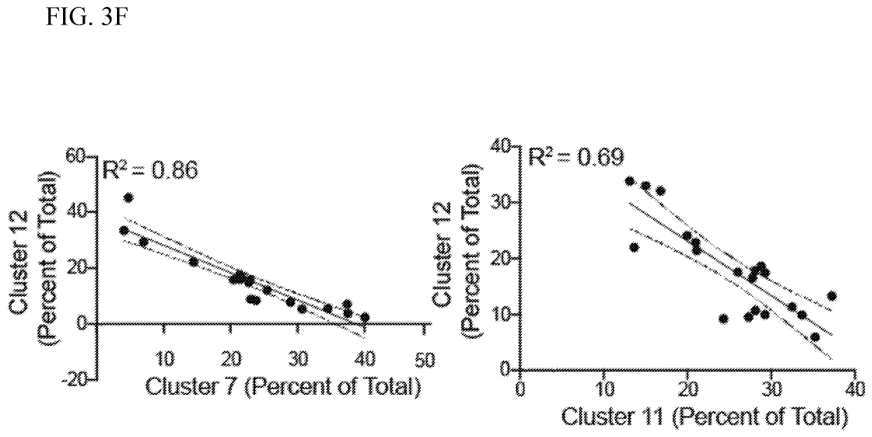

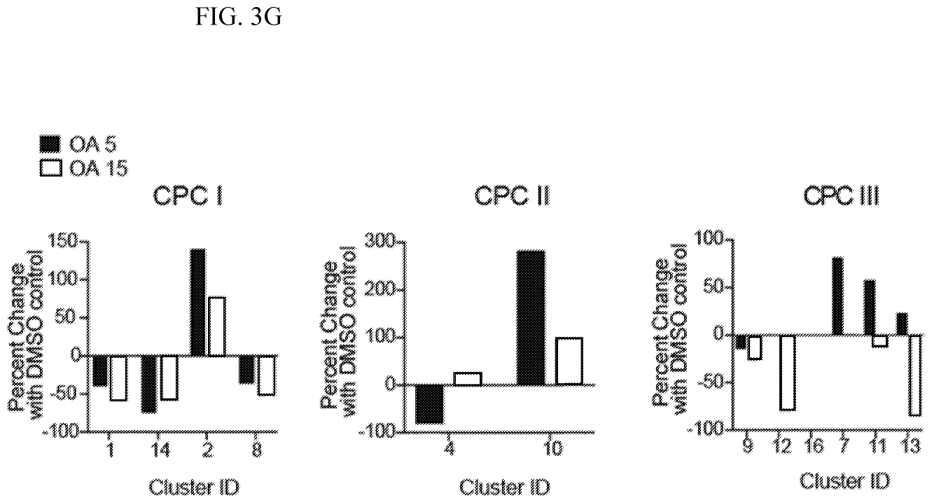

Several studies have found cartilage-progenitor cells (CPCs) that have the ability to give rise to chondrocytes, show self-renewal in culture, and have high migratory ability in OA cartilage (see for examples Refs. 3, 4, 11-18). These CPCs are believed to be the origin of the highly clonal characteristic clusters found in OA cartilage (see for examples Refs. 19, 20). Their role in OA disease pathology, however, remains unclear, especially whether they contribute to disease onset and progression. To address these questions and better characterize the CPCs and their crosstalk with other cartilage resident cells, the cyTOF panel was had designed to include 13 previously described markers for CPCs (Table 2, FIG. 3A). Of the 20 clusters identified using flowSOM, 12 clusters were found to be positive for these CPC markers in a variety of combinations (FIG. 3A). In contrast to previous observations, data showed that there were four novel variants of these CPC subpopulations that are depleted in OA (FIG. 3B), which was termed CPC I. Out of the rest, two clusters were unchanged between normal and OA cartilage, termed CPC II, and six clusters were enriched in OA cartilage, comprising some of the previously described CPC populations, which was termed CPC III (FIG. 3B).

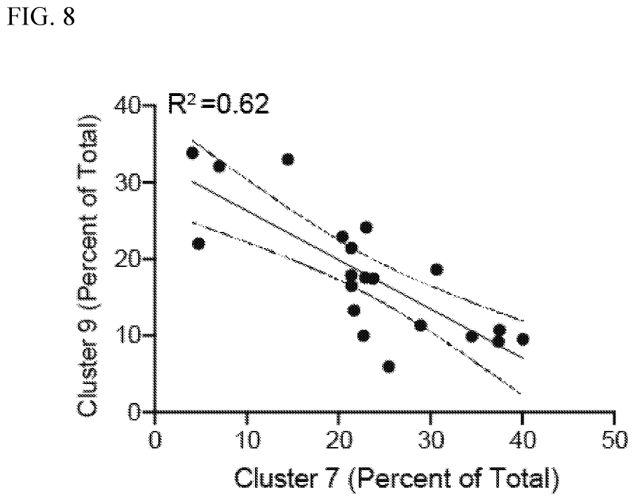

The CPC I clusters were characterized by lower CD105 expression in contrast to the CPC III clusters (FIG. 3A). Cluster 1 and 2 cells were distinct in having a high expression of CD54 (ICAM) (FIG. 3A). Previous work exploring markers for stem or progenitor cells had noted that cells with high CD54 and CD55 expression had higher levels of ALDH activity, associated with stem cell function (See for example Ref. 21). Cluster 14 was distinguished by the expression of CD151 i.e. tetraspanin, a cell adhesion marker, which was described to mark chondrocytes with higher chondrogenic potential in an in vitro study (See for example Ref. 22). Cell cycle analysis showed that CPC I clusters had the highest percentage of cells that were cycling (FIG. 3C), though overall the number of cycling cells was low as expected for post-mitotic chondrocytes (<20%). The CPC I clusters are exclusively characterized by ERK1/2 signalling while the other clusters, with the exception of the CPC II cluster 10, are not (FIG. 3D). Out of the CPC II clusters, cluster 4 is characterized by a high CD73 expression and is not predominantly active in any of the tested signaling pathways (FIG. 3D). CD73 has recently been identified to be one of the critical markers on an adult human skeletal stem cell population (hSSC) (See for example Ref 5). Intriguingly, both highly inflamed and non-inflamed clusters were present among the OA enriched CPC III populations. Clusters 12, 13 and especially 16 were high in the expression of inflammatory markers, such as pNFK-B, pSTAT3, BCAT and HIF2A, while clusters 7, 9 and 11 were low in inflammation (FIG. 3D). Cluster 16 appears to be the quintessential CD105/CD90 high, NOTCH1-1/STRO-1 driven migratory CPC that has been previously identified in OA cartilage (See for example Ref 14, 23). Group C patients had a significantly higher percentage of the proinflammatory clusters 9, 12 and 16 and a lower percentage of low-inflammation clusters 7 and 11 (FIG. 3E). This anti-correlation between clusters 9 and 11, clusters 12 and 7 and clusters 16 and 14 (FIGS. 3F and 8) held across the 20-patient cohort, suggesting that these patients might be particularly driven by this cellular subtype.

Experiments were undertaken to understand how the application of a pro-regenerative drug would affect this CPC landscape, specifically the pro-inflammatory CPCs four in Group C. Kartogenin (KGN) was originally identified in a screen to expand mesenchymal stem cells and has since been shown in multiple studies to be a pro-chondrogenic modulator of OA progression in animal models (See for example Ref. 124-26). One patient from Group C (OA15) and one patient from Group A (OA5) were utilized. One patient from Group A (OA5) and one patient from Group C (OA15) were treated with kartogenin or control (DMSO) for 48 hours, fixed, stained and profiled by cyTOF as previously described. Kartogenin treatment selectively expanded cluster 2 (CPC I) and cluster 10 (CPC II) at the expense of the other CPC I and II clusters, in both the patients (FIG. 3G). The treatment additionally expanded low-inflammation clusters 7 and 11 in the Group A patient, where they already appeared overrepresented, while these clusters were not expanded in the Group C patient. Furthermore, kartogenin reduced pro-inflammtory clusters 9, 12 in both the patients, though to a greater degree in the Group C patient enriched in those subtypes (FIG. 3G). Overall, kartogenin appears to expand normal-like and low inflammation CPC clusters (2, 10, 7 and 11) while reducing the high inflammation CPC III clusters (12 and 16).

Identification of a Rare Inflammation Amplifying Population in OA Cartilage

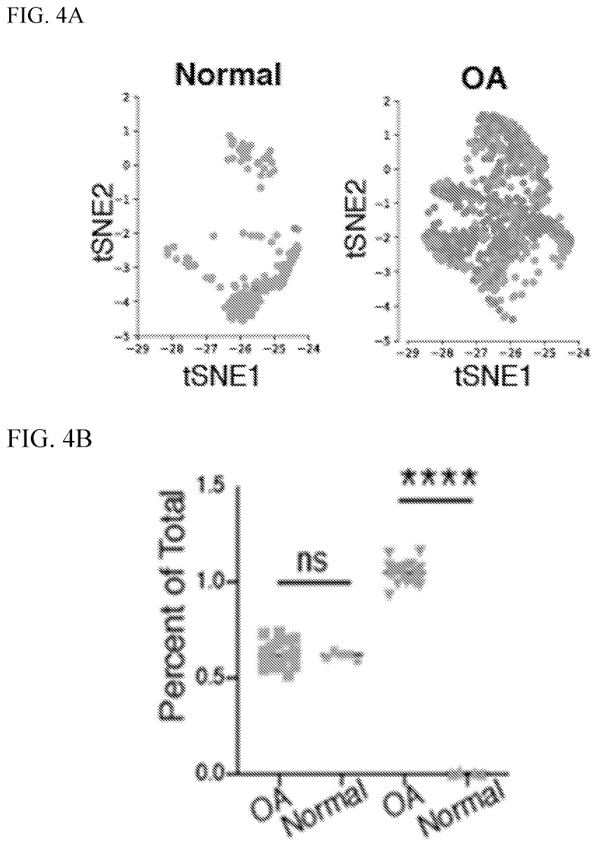

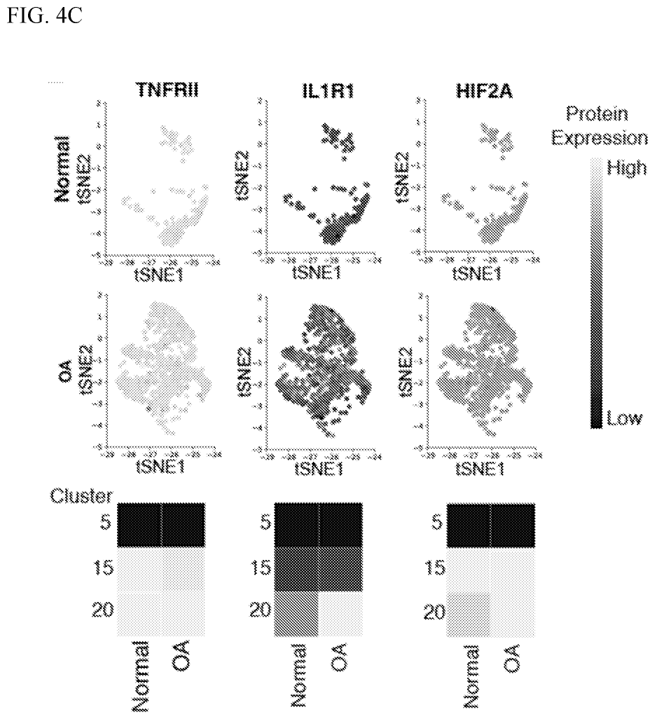

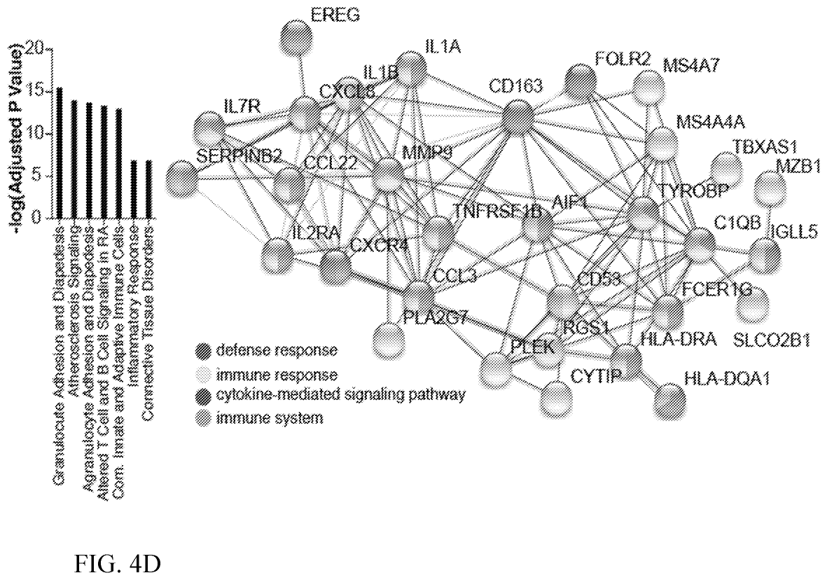

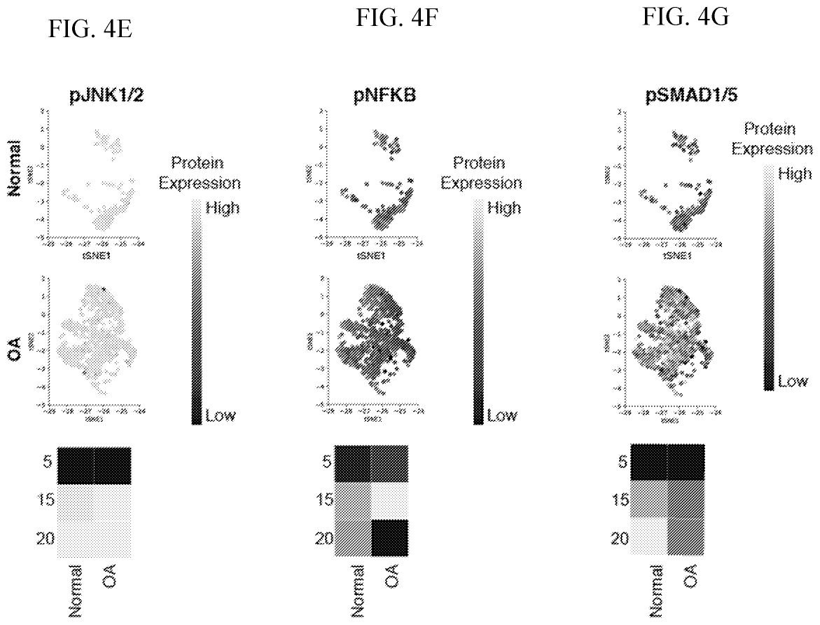

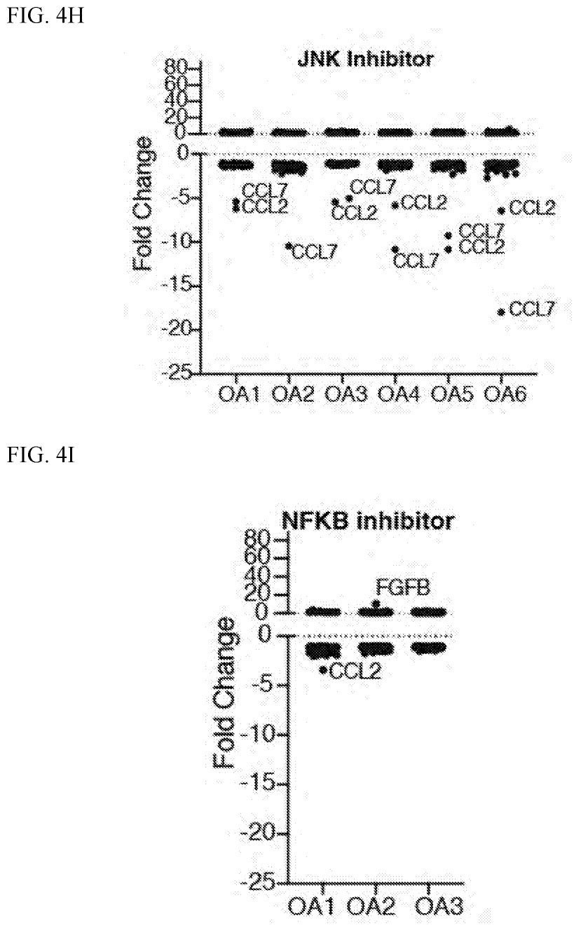

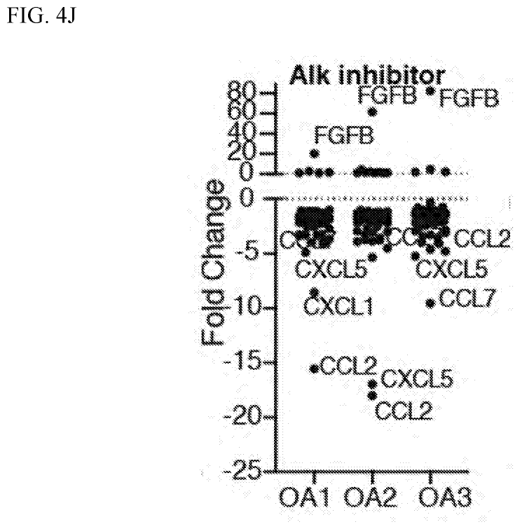

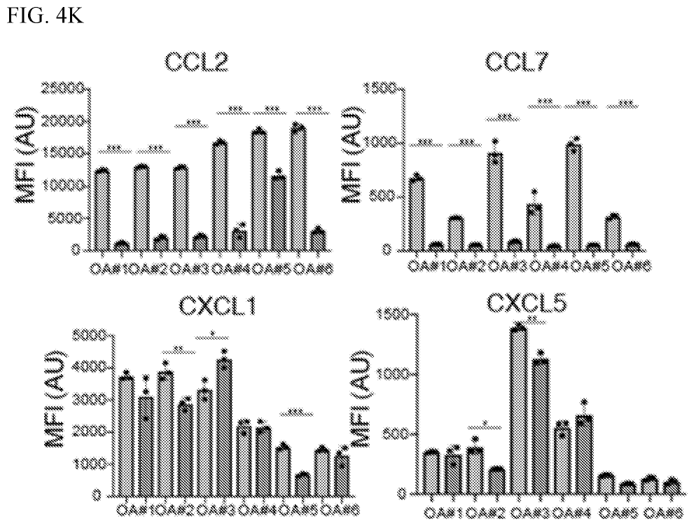

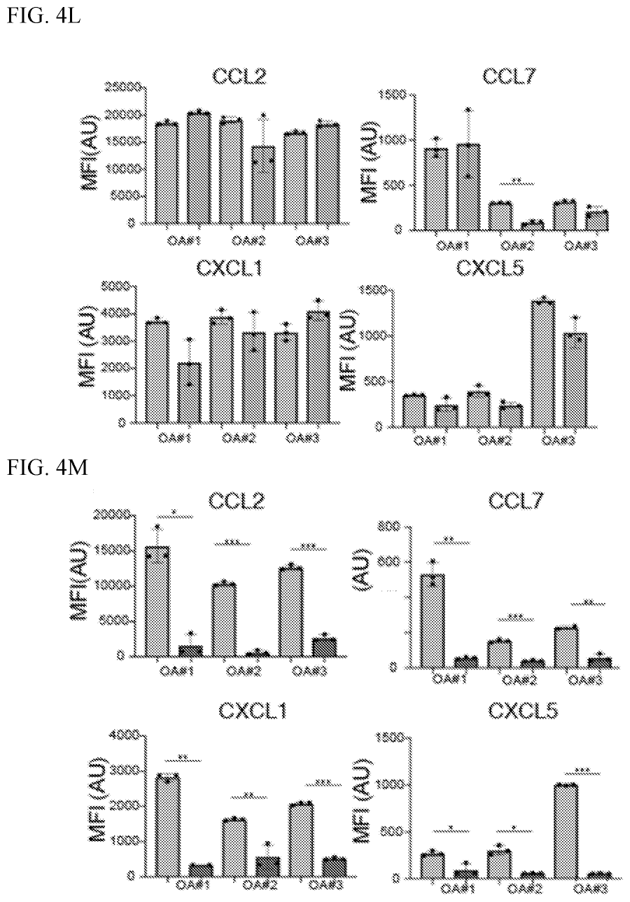

The non-CPC populations that were identified by the panel were further analyzed, with a focus on putative inflammatory populations that might contribute to pathology. Among these were Clusters 15 and 20, which are characterized by the co-expression of two cytokine receptors, IL1R1 (CD121A) and TNFRII (CD120B) (FIG. 4A). Cluster 20 is significantly expanded in OA cartilage compared to the normal cartilage (FIG. 4B). Clusters 15 and 20 vary in the quantity IL1R1 expression, with cluster 20 having a higher level of IL1R1 (FIG. 4C). However both clusters 15 and 20 have similarly high levels of TNFRII and HIF2A expression (FIG. 4C).

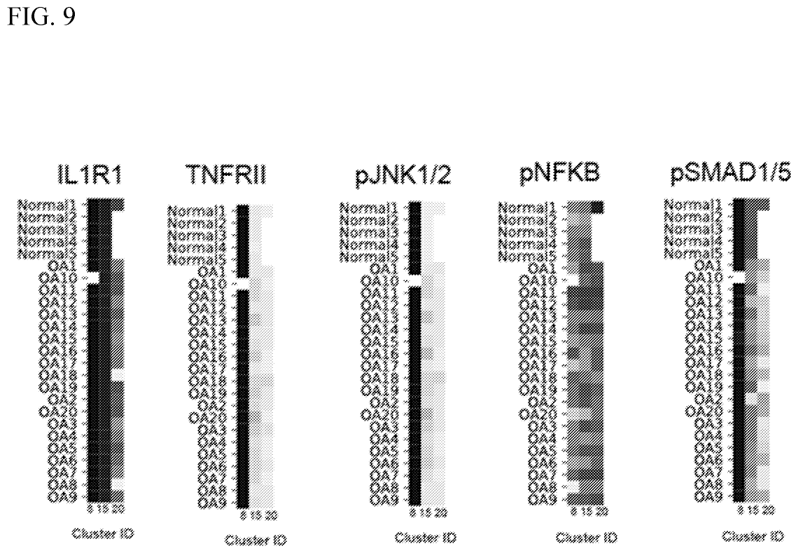

To further understand the molecular underpinnings of these subpopulations, publicly available single-cell (sc)-RNA sequencing data was utilized (See for example Ref. 27). Chondrocytes that expressed both IL1R1 and TNFRII transcripts were sorted in silico and the differentially expressed genes and pathways were analyzed. The IL1RI/TNFRII expressing chondrocytes were found to be highly enriched in pathways related to innate and adaptive immune cells, inflammation and altered T and B cells signalling in arthritis (FIG. 4D). These analyses suggested that the IL1RI/TNFRII cells might act to recruit immune cells to the joint space. The clusters 15 and 20 were termed inflammation amplifying chondrocytes (Inf-A). Upon analyzing their signalling status, the Inf-A clusters showed exclusive signalling through pJNK and pSMAD1/5 compared to the rest of the chondrocyte clusters (FIGS. 4E and 4G). In contrast, pNFK-B levels in clusters 15 and 20 were similar to other clusters identified (FIG. 4F). Despite its rarity, cluster 20 was highly consistent among patients, with TNFRII expression and JNK and SMAD1/5 phosphorylation levels consistently high across all OA patients in cluster 20, and more variable in cluster 15 (FIG. 9). Indeed, cluster 20 showed the lowest coefficient of variation in the OA samples (FIG. 2E).

Next, experiments were conducted to explore the functional effects of inhibiting these newly identified Inf-A cells in OA cartilage by capitalizing on their distinct signalling through JNK. Chondrocytes derived from 6 patients were cultured for 48-hours in the presence of JNK II inhibitor and the secretome was analyzed via 62 antibody human Luminex panels. Across all six patients, a variety of cytokines were altered, many trending toward significance. Restricting the analysis to only those cytokines that were altered in 5 or more patients (>83% response rate), data demonstrated a significant decrease in CCL2 and CCL7 after JNK inhibition (FIGS. 4A-4M). CCL2 and 7 are well-established chemoattractants for monocytes and are known to be altered during OA progression (see for example Ref 28). Genetic deletions of CCL2 and its receptor CCR2 prevent the development of surgical OA, further underscoring the importance of CCL2 as a key modulator in pathology (see for example Ref. 28). In contrast, inhibition of NFKB activity with BMS-345541 did not affect CCL2 or CCL7 secretion in OA chondrocytes (FIGS. 4I and 4L), suggesting the effect is specific to the Inf-A population (See for example Ref 30). As a complementary approach, SMAD1/5, the other exclusive signaling pathway of the Inf-A cells was inhibited using an ALK inhibitor. ALK receptors are the most common upstream target of SMAD1/5 signaling in OA (See for example Ref. 31). As hypothesized, ALK inhibitor treatment resulted in a decrease of the same cytokines affected by the JNK inhibitor, CCL2 and CCL7, and additionally CXCL1 and CXCL5 (FIGS. 4J and 4M) two other leukocyte attracting factors. Collectively, these data were consistent with the transcriptional data suggesting that the IFNR1/TNFRII co-expressing cells mark a rare and novel OA subpopulation that is potentially responsible for immune recruitment to the joint. Data herein demonstrated that inhibition of this rare population can significantly affect the overall secretome of the end-stage OA chondrocytes.

A CD24⁺ Chondrocyte Population Mitigates Inflammation in OA Cartilage

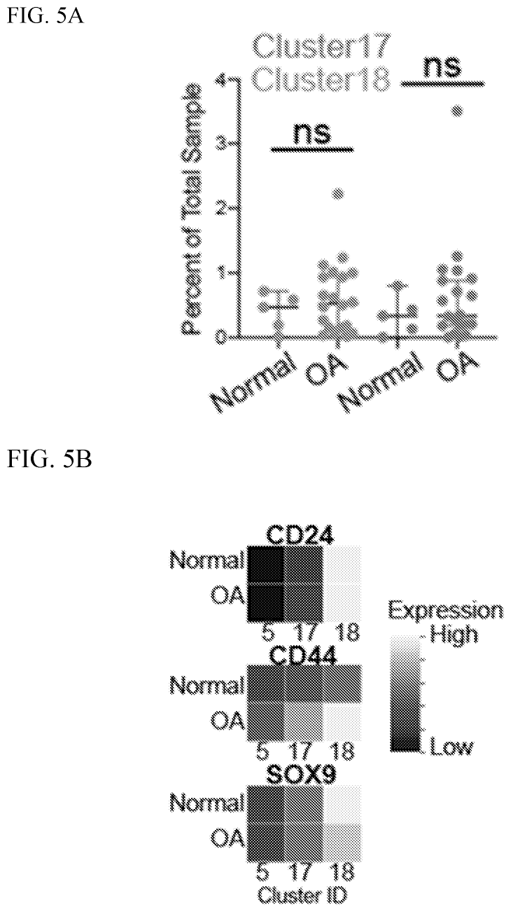

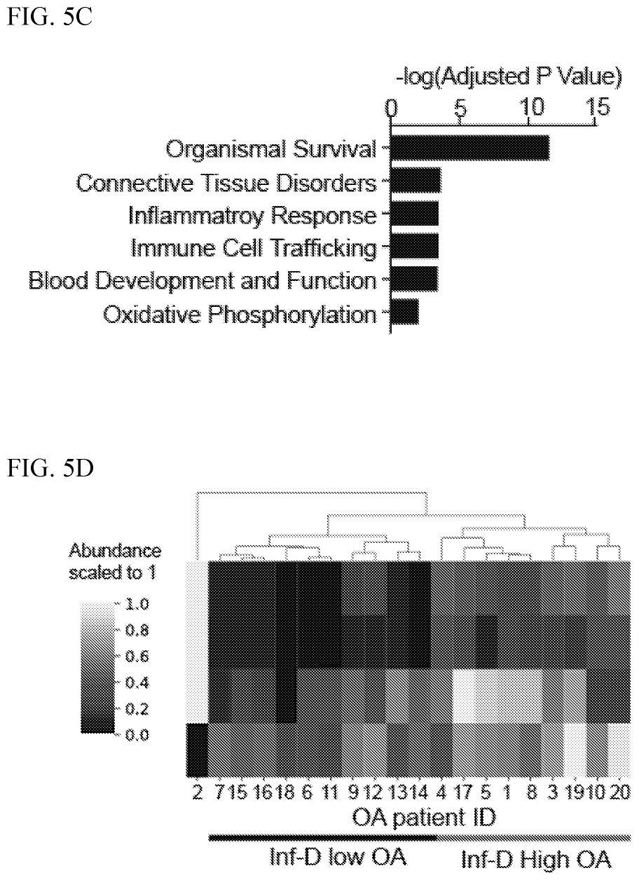

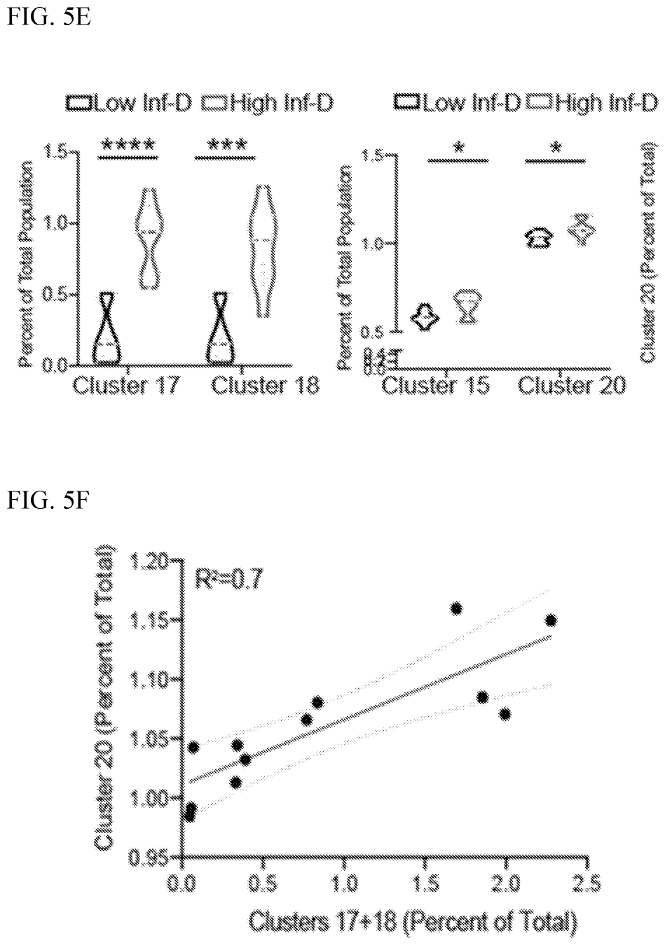

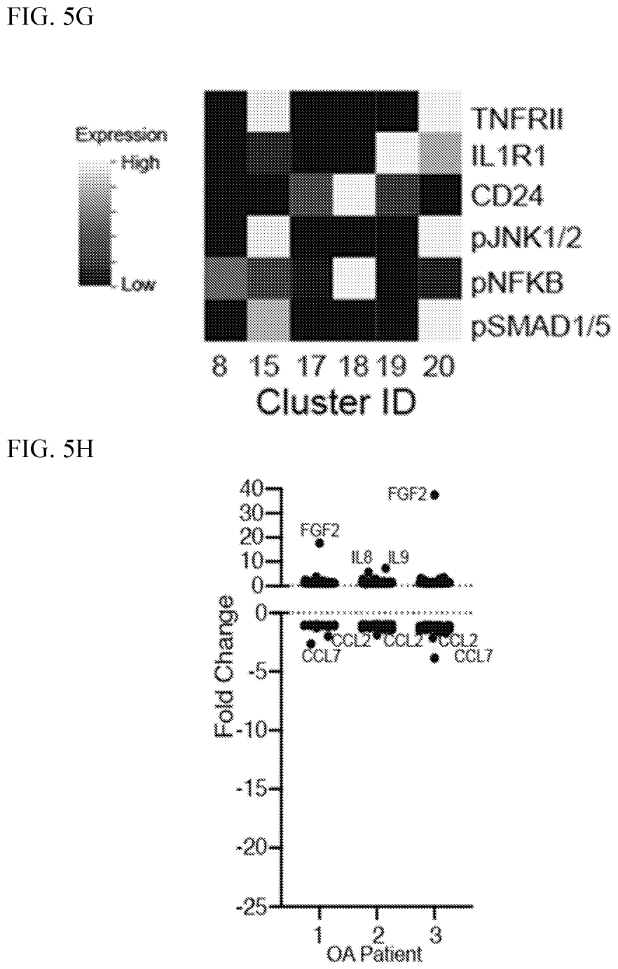

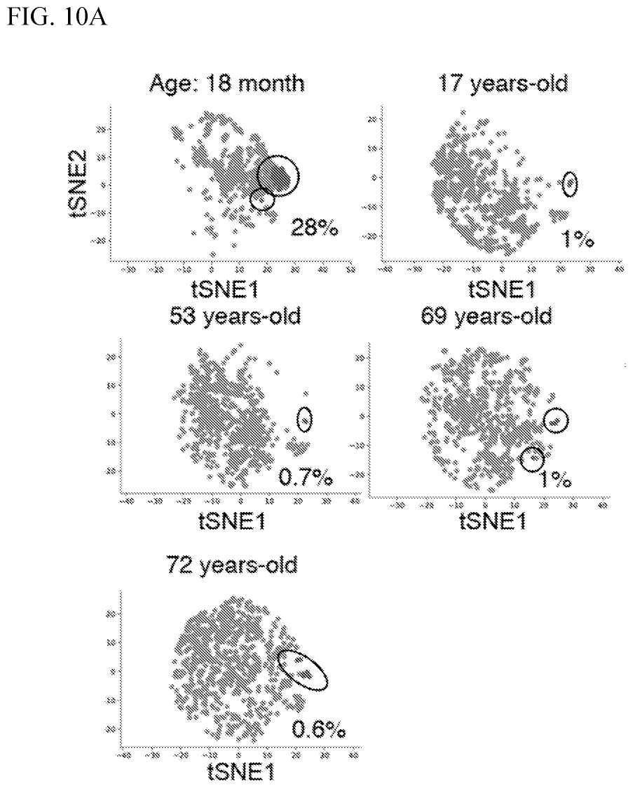

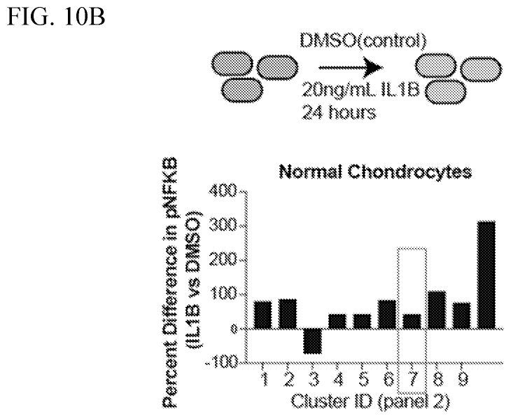

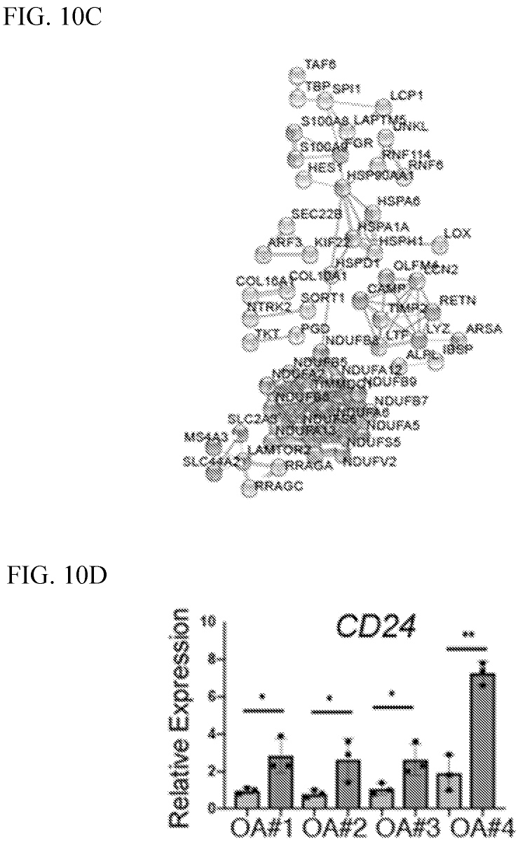

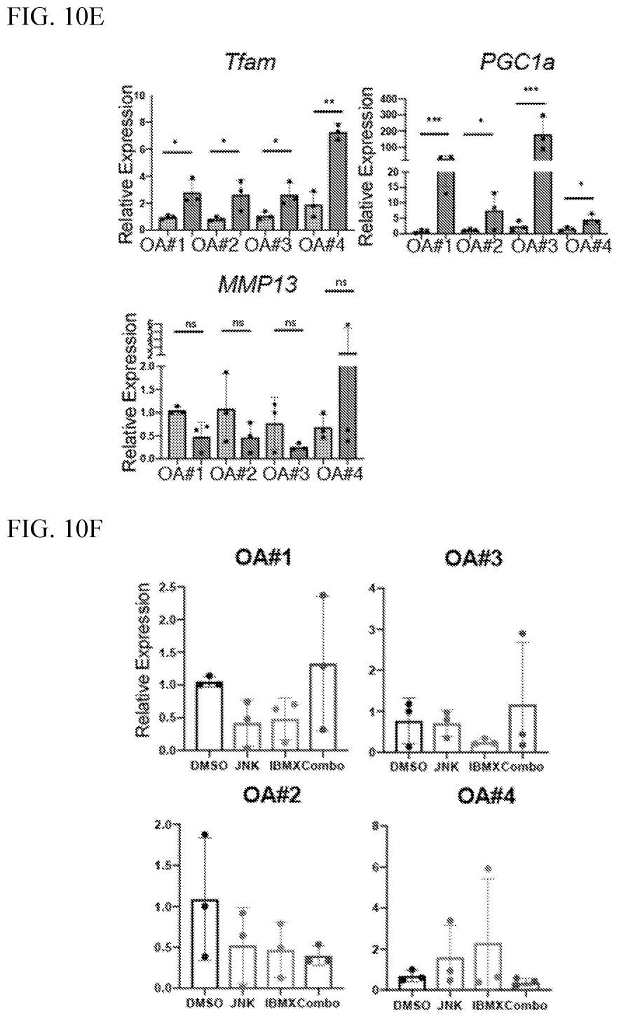

Previous work established a role for the cell surface receptor CD24 in mitigating inflammation in healthy and induced pluripotent stem cells (iPSC)-derived chondrocytes (See for example Ref. 32). Although CD24 is highly expressed in juvenile and iPSC derived chondrocytes, its expression is decreased with age, potentially underscoring the age-related etiology of OA. CD24 was included in the cyTOF panel to understand the interplay of CD24⁺ cells with the other regenerative and inflammatory subpopulations in the OA joint. FlowSOM derived clusters 17 and 18 were found to be most enriched in CD24 expression (FIG. 5B). Both clusters 17 and 18 were found in equal numbers in normal and OA cartilage, however there was a high variability in their abundance between patients (FIG. 5A). In agreement with previous work, CD24 cells decreased with age (FIG. 10A) and were among the least reactive groups to undergo stimulation by the pro-inflammatory cytokine IL1B (FIG. 10B). Therefore, clusters 17 and 18 were termed inflammation dampening cells (Inf-D) I and II respectively. Inf-D II cells had the highest levels of CD24 expression, and also had higher levels of Sox9 and CD44, though expression in Inf-D I cells was comparable with normal cells (FIG. 5B). To further characterize the function of these CD24⁺ cells, the same previously published scRNA-seq data set was used and sorted out CD24 positive cells. Consistent with the hypothesis that the CD24⁺ cells are capable of immune modulation, an enrichment for pathways related to inflammation and immune cell trafficking and cross-talk was observed (FIGS. 5C and 10C). In addition, the CD24⁺ cells showed an enrichment of oxidative phosphorylation pathways, suggesting that these cells could have different metabolic processes compared to other chondrocytes (FIGS. 5C and 10C).

To understand the interplay between Inf-A and Inf-D cells in the OA cartilage, their abundance was analyzed in the cohort of 20 patients and hierarchical clustering was used to order patients by the content of their Inf-A and Inf-D cells. The patients were clearly stratified into two large categories of patients: Inf-D low and Inf-D high OA patients (FIG. 5D). The Inf-D high group had concomitantly high levels of the Inf-A clusters than the Inf-D low group (FIG. 5E). Additionally, a positive correlation was observed between the percent of Inf-A and Inf-D cells in patients (FIG. 5F). This led to a hypothesis that a combination strategy of enhancing Inf-D while inhibiting Inf-A populations could be effective in mitigating inflammation in OA cartilage. This hypothesis was further strengthened by the presence of another small and highly variable population, cluster 19, which had a mixed character. Cluster 19 showed IL1R1 expression without the inflammatory signature that was observed in the Inf-A I and InfA-II cells (pJNK1/2 and pSMAD1/5) (FIG. 5G) and curiously also expressed CD24. These cells were only present in eight out of the 20 patients (FIG. 5G), but further suggested that CD24 expression in the Inf-D cells can dampen inflammation.

To test this hypothesis, first induced mild CD24 overexpression was introduced by treating cells with IBMX, a cAMP inhibitor that has been shown to increase CD24 expression in adipocytes (See for example Ref. 33). Treatment with 0.5 mM IBMX for 48 hours upregulated CD24 expression by 2-4 fold in OA chondrocytes (FIG. 10D). IBMX increased the gene expression of the mitochondrial genes, Tfam and Pgc1a (FIG. 10E), though no consistent effect was however observed on MMP13 expression (FIG. 10E). Using the 62-plex Luminex array, data showed a modest downregulation of CCL2 and CCL7, however these effects were milder than the direct inhibition of the Inf-A signaling (FIG. 5H).

Then, a combination treatment of JNK inhibitor with IBMX for 48 hours was tested. Data showed a greater magnitude decreased in CCL2 and CCL7 with the combination treatment (FIG. 5I) as compared to the single treatment with JNK inhibitor (FIGS. 4H and 4K). In addition, the combination therapy further mitigated inflammation by reducing the secretion of new targets like IL21, IL22, VCAM and IFNB1 (FIG. 5L). Similar to JNK inhibitor treatment, the MMP gene expression remained unaffected by the combination treatment (FIGS. 10A-10F). These data, however, confirmed the interplay between the Inf-A and Inf-D populations and suggest that targeting multiple combinations of rare cell types in OA cartilage may be beneficial in mitigating inflammation.

Discussion

Experiments herein built the first single-cell, proteomic atlas for healthy and osteoarthritic adult articular cartilage. Cartilage regeneration and OA remain unmet medical needs. Therefore, a high-resolution cellular atlas of articular cartilage tissue lays the foundation for insight into disease pathology, new drug strategies and tissue engineering. Using a panel of 33 markers, multiple populations were identified that constitute the articular cartilage landscape, including rare populations that contribute to disease pathology and inter-patient heterogeneity.

Recently, a single-cell RNA-sequencing map of cartilage tissues was reported from a cohort of ten OA patients, that outlined several known and novel cell populations in OA cartilage (See for example Ref 27). The study compliments this single-cell transcriptomic data, with the additional advantage that the proteomic snapshot provides status of signalling pathways in the identified subpopulations. The single-cell proteomic approach is especially pertinent in robustly identifying rare cell populations that are difficult to discern from RNA-sequencing data, where only 1600 cells were studied from all the OA patients. In contrast, the ability to map 30,000 to 100,000 cells per patient in a 20 patient cohort by the cyTOF method provided a robust dataset to find and validate statistically significant rare subpopulations. Indeed, a recent study on rare senescent cell populations in OA cartilage has shown the influence of such small populations in OA pathology (See for example Ref. 34). Removal of senescent cells significantly impaired OA progression in a mouse model and modulated end-stage human OA chondrocytes, underscoring the need for further studies on other rare populations that might contribute to OA pathology. In addition, frequent discrepancies between gene and protein expression have been reported in OA further signifying the need for complementary proteomic and transcriptomic studies at both population and single cell level.

The ability to measure a large number of cells with high precision allowed identification of two novel, rare chondrocyte subpopulations (Inf-A and Inf-D), which constitute only 0.5-1.5% of all chondrocytes. However, pharmacologically targeting these small populations led to a significant dampening of inflammation at the population level. The inflammation amplifying (Inf-A) cells express both the TNFR II and IL1R1 receptors, are consistently expanded in OA compared to normal cartilage, and are characterized by activated JNK1/2 and SMAD1/5 pathways. An analysis of their transcriptomes from the published single cell RNA-seq dataset suggested that these cells may function to recruit immune cells. Inhibition of these cells using a INK inhibitor led to an overall reduction of secreted CCL2 and CCL7, cytokines implicated in immune cell recruitment (See for example Refs. 35, 36). Genetic knockout of JNK1 or JNK2 ameliorates disease symptoms in a collagenase induced model of RA (See for example Ref. 37) and inhibition of JNK protects joints from characteristic degeneration (See for example Ref. 38). However, unlike in RA models, JNK inhibitors have not been systematically studied as a therapy in animal models of OA. TNFRII antibodies also have a strong therapeutic index in RA (See for example Ref. 39). The work herein suggests that some of these therapies may also be successful in targeting OA.

The other novel population identified in this study was the inflammation-dampening (Inf-D) chondrocytes, which are characterized by the expression of CD24, a cell surface receptor previously reported to be enriched in juvenile cartilage and associated with resistance to inflammatory cues (See for example Ref. 32). Intriguingly, expression of CD24 in Inf-A cells, a subpopulation observed in some patients, led to a complete inhibition of JNK activation. In addition, the positive correlation between Inf-A and Inf-D populations in a subset of patients supported a hypothesis of an interplay between these two populations. Combinatorial treatment with JNK inhibitor (lowering Inf-D) and IBMX, a small molecular activator of CD24 (increasing Inf-D) showed a greater decrease in CCL2, CCL7, CXCL1, CXCL5 and other inflammatory cytokines than JNK inhibition alone. The data therefore provided insights into the interplay between multiple cellular populations that likely contribute to the chronic inflammatory environment that is observed in end-stage OA cartilage. A deeper understanding of these populations, their cross-talk, and relative influence can help devise single or combinatorial biologic candidates that can tilt the inflammatory balance in a way that can be beneficial in the later or early stages of OA progression.

The data herein also served to redefine the cartilage stem and progenitor-like populations that reside in adult cartilage. The existence of CD105/CD90, NOTCH1, STRO1 expressing CPCs that have been previously described in OA and are highly inflammatory was validated. Additionally, described herein are other CPC populations in OA cartilage that express CD90 and CD105 but are low in inflammation. It will be interesting to compare the regenerative potential of these different subpopulations of CPCs, especially in a low inflammation microenvironment. Since CD24 is a marker for younger chondrocytes with a higher regenerative potential, it is possible that the combinatorial treatment can boost regenerative populations in addition to mitigating inflammation. The data also revealed that CD24 expression is associated with mitochondrial biogenesis, another characteristic associated with younger healthy chondrocytes. The data also revealed CPC I as progenitor populations that are lost in OA. Future studies are needed to determine how these CPCs are lost during OA progression and whether reintroduction of these CPCs can benefit cartilage regeneration. A particularly interesting subgroup to follow is the CD73 expressing cells, as CD73 has recently been identified to characterize the human skeletal stem cells (hSSC) in bone marrow, which can self-renew and give rise to cartilage, bone and fat progenitor cells (See for example Ref 5).

By characterizing chondrocyte populations in OA patients, patients were stratified by the abundance of each population. This practice is well established in the cancer field, where patient heterogeneity and tumor subtyping play an ever increasing role in the precision medicine. Identification of the 20 different subpopulations in cartilage revealed three major categories of OA patients. Group A represents 60% of the patients while Groups B and C represent 15% each. Group C patients were distinguished from Group A and B patients by expansion of the inflammatory Notch-1/STRO-1 expressing CPCs, which are also highly active in pro-inflammatory pathways such as NFKB and HIF2A. Group B patients had an expansion of the Inf-D population. A subset of patients driven by inflammation has been suggested previously as well based on RNA-sequencing and DNA methylation patterns in cartilage (See for example Refs. 40-42).

In summary, this study provided the first high dimensional cyTOF map for adult cartilage, revealing multiple, rare subpopulations that coexist in health and disease. Collectively, the data highlighted the complex interplay between inflammation amplifying and dampening populations and regenerative populations in cartilage and suggested that altering the balance between these populations could provide novel therapeutic strategies for OA. In future studies, refined panels and larger cohort sizes can provide a powerful platform for the stratification of OA patients based on the underlying cellular drivers of their disease. Ultimately, such stratification efforts would allow for targeted testing of drugs for each patient subset, to establish personalized medicine strategies for OA.

### Example 3: Methods

Study Design

Research objectives: The objective was to profile rare populations of cartilage-progenitor cells in OA patient samples and determine their interactions. A curated panel of antibodies (see below) was designed and used to test a cohort of 20 OA patients and 5 normal samples. Observations from this data set were then more thoroughly tested. Research subjects: Chondrocytes were derived from OA cartilage or healthy cartilage samples. All experiments were performed on primary cells. Experimental design: A cohort of 20 patients was collected, which passed several quality control parameters (see below) and included a variety of ages and balanced pool of male/female patients. Samples which did not pass quality control metrics were not utilized for downstream analysis. Patient samples were selected based on previously established QC criteria, namely the expression ratio of Col2a1 Col1a1 (see methods below) and the expression of MMPs. Follow up analysis was conducted on a separate panel of OA chondrocytes to ensure that one could see the same results independently. Blinding: Researchers were not blind to disease status or treatment when analyzing the data. Data inclusion exclusion criteria: All collected data points were utilized for assays performed after drug treatment. All data sets were quality controlled, and wells or data points that did not pass quality control metrics did not get utilized. This included: Luminex wells that did not give acceptable standard bead readings, qPCR wells that did not give suitable Ct values for Actin, cells analyzed by cyOF that did not have high SOX9 or CD44 expression. Quality control exclusions were performed prior to analysis of data. After exclusion of points for these reasons, no additional points were excluded. Replicates: All drug treatments were performed in independent technical replicates for each patient (i.e. cells derived from the same patient were treated 3 times with drug versus control). All drug treatments were performed in 3-6 patient samples.

Isolation and Culture of Primary Chondrocytes from Human Cartilage

OA samples were procured from the discarded tissues of patients with radiographic OA undergoing total-joint replacement, in accordance with the IRB protocol approved by Stanford University, as previously described (See for example Ref. 9). The age range for OA patient samples was 54-72 years old. Cartilage was shaved from the underlying bone, allowed to recover overnight at 37° C. in complete media (Hyclone DMEM:F12 (GE Healthcare, SH3002302) supplemented with 2 mM L-glutamine (Gibco, 25-030-149), 10% FBS (Corning 35-016-CV), 1× Antibiotic-Antimycotic (Gibco, 15-240-062) and 12.5 μg/mL ascorbic acid (Eastman)) and then treated with collagenase (2.5 mg/mL each Collagenase II and IV (Worthington Biochem)) in complete media overnight at 37° C. The next day, cells were strained, centrifuged and plated at a high density of 2.6×10⁴ cells/cm in complete media. Cells were allowed to become confluent on the plates and were passaged once using collagenase, prior to cyTOF experiments or drug treatments. Samples were checked for Col2a1/Col1a1 ratios and MMP 3, 9 and 13 expression, prior to experimentation. Normal samples were either derived from expired cartilage allograft samples, shipped from the manufacturer (samples 1-4) or from the surgical waste of a notchplasty (sample 5) under an approved IRB, and processed as described above.

RNA Isolation and cDNA Synthesis

Cells for RNA extraction were collected in RNA lysis buffer (Zymo Research) and processed according to the manufacturer's specifications for the Quick-RNA MicroPrep Kit (Zymo Research, R1051), including the optional DNAse1 digestion. RNA quality and quantity was measured using the Nanodrop 1000 Spectrophotometer. All samples had A260/280 scores between 1.6-1.8.

Gene Expression Analyses

One mg of RNA from each sample was reversed transcribed into cDNA using the High Capacity cDNA Reverse Transcription Kit (Applied Biosystems, 4368813). Quantitative PCR was performed using TaqMan gene-specific expression assays, FAM-labeled, for metalloproteinase 3, 9, a 13 (Hs00233962_m1, Hs00957562_m1, Hs00233992_m1), with a universal mastermix (Applied Biosystems, 4369016). Gene expression levels were normalized with FAM-labeled Actin-beta (Hs01060665_g1).

For Tfam, CD24 and PGC1a, we utilized the SybrGreen mastermix (Applied Biosystems, A25742) according to manufacturer's specifications. Primer sequences: Tfam_F: 5′-GCTCAGAACCCAGATGCA AAA-3′, Tfam_R 5′-AGGAAGTTCCCTCCAACGC-3′, PGCla_F: 5′-CCATGGATGAAGGGTACTTTTCTG-3′, PGC1a_R: 5′-CTTTTACCAAAGCAGCAGCC-3′, CD24_F: 5′-TACCCACGCAGATTTATT-3′, CD24_R: 5′-AGA GTGAGACCACGAAGA-3′, Actin_F 5′-CACCAACTGGGACGACAT-3′, Actin_R: 5′-ACAGCCTGGATAGCAACG-3′. qPCR reactions included a 2-minute incubation at 50° C. to inactivate previous amplicons with uracil-DNA glycosylase, followed by a 10-minute incubation at 95° C. to activate the Taq polymerase. The amplification cycle, consisting of 15 seconds at 95° C., and 1 minute at 60° C., was repeated 40 times. The relative expression levels were determined using the ΔCt method (CT gene of interest−CT internal control−Actin) and relative gene expression is calculated using 2−ΔCt method and plotted.

Drug Treatment of OA Cells

OA cells were seeded at high density in 12-well plates and treated with control (DMSO) or drug next day for 48 hours. Drug doses were determined based on prior literature and validation-: 0.5 mM 3-Isobutyl-1-methylxanthine (IBMX, Sigma 15879) (See for example Ref. 33), 50 μM JNK Inhibitor II (Calbiochem 420119), 25 μM NFK-B inhibitor BMS-345541 (Sigma B9935) (See for example Ref 30, 43), 50 μM Alk inhibitor, SB 431542 hydrate (Sigma S4317 (See for example Ref. 44, 45) and 25 uM Kartogenin (Sigma SML0370) (See for example Ref. 24-26) were used with appropriate dilution in DMSO.

Multiplex Autoantibody Assay

Cell culture supernatants were collected and spun down at 10,000×g for 10 min at 4° C. to remove any cells or cell debris and then snap frozen in liquid nitrogen, before performing the assay. This assay was performed in the Human Immune Monitoring Center at Stanford University. Human 62-plex kits were purchased from eBiosciences/Affymetrix and used according to the manufacturer's recommendations with modifications as described below. Briefly, beads were added to a 96-well plate and washed in a Biotek ELx405 washer. Undiluted samples were added to the plate containing the mixed antibody-linked beads and incubated at room temperature for 1 hour followed by overnight incubation at 4° C. with shaking. Cold and room temperature incubation steps were performed on an orbital shaker at 500-600 rpm. Following the overnight incubation plates were washed in a Biotek ELx405 washer and then biotinylated detection antibody added for 75 minutes at room temperature with shaking. Plate was washed as above and streptavidin-PE was added. After incubation for 30 minutes at room temperature wash was performed as above and reading buffer was added to the wells. Each sample was measured in duplicate. Plates were read using a Luminex 200 instrument with a lower bound of 50 beads per sample per cytokine. Custom assay Control beads by Radix Biosolutions were added to all wells.

Conjugation of Antibodies to Metal Isotopes

Antibodies were labeled according to the manufacturer's specifications using the MAXPAR X8 Polymer labeling kit (Fluidigm). One tube of was used per 100 ug of antibody. Antibodies were purchased labeling ready, without additives, whenever possible. Antibodies with carrier components such as albumin or glycerol were cleaned with Melon Gel IgG Purification columns (Thermo Scientific) after buffer exchange with Zeba Desalt Spin Columns (Thermo Scientific) as per the manufacturer's specifications. Final antibody concentration was measured using a Nanodrop 1000 Spectrophotometer, set to IgG mode and diluted to the highest round value in W buffer with sodium azide and stored at 4° C. for later use. The complete list of conjugated antibodies, metal isotope, clone information and manufacturer can be found in Table 2.

Titration of Antibodies for cyTOF

Metal conjugated antibodies were tested in a three point dilution curve, centered on their recommended or optimized FACS sorting concentration, with a 10-fold increase and decrease from this center value. Signal to noise ratio was compared by staining known negative samples, such as 293T cells. The lowest concentration that had no increase in signal upon a 10-fold increase in concentration was used for the final staining concentration (see Table 2).

Cell Staining and cyTOF

OA cells were cultured to confluence in 10 cm dishes. On the collection day, cells were stained with 25 μM Idu for 15 min at 37° C. in the cell incubator, then with 0.5 μM cisplatin for 5 min at RT. Cells were then lifted with 0.25% Trypsin-EDTA (Gibco) for 15 min at 37° C. Trypsin was quenched using media containing 10% FBS and cell were washed 3 times with PBS to remove any trace amounts of trypsin. Cells were fixed after straining through a 35 μM strainer in 1.6% PFA for 10 min at RT. Cells were washed 4 times with cells staining media, counted and frozen in 1 million cell aliquots in a small amount of cell staining media at −80° C. To stain, cells were thawed on ice and barcoded using the Cell-ID 20-plex Pd Barcoding Kit (Fluidigm) according to the manufacturer's specifications. After barcoding, cells were labeled as previously described (See for example Ref 13). Briefly, all barcoded samples were combined into one FACs tube, and washed 3× with cell staining media and stained with the cell surface antibodies for 30 minutes at RT according to the concentrations in Table 2. Cells were then was 2× with cell staining media and permeabilized with 1 mL of cold methanol added dropwise with continuous gentle vortexing. Cells were incubated for 10 min on ice, with gentle vortexing every 2-3 minutes to avoid cell clumping, then washed in cell staining media and stained with the intracellular antibodies for 30 minutes at RT. After 2× washed with cell staining media, cells were resuspended in 1.6% PFA with Cell-ID Intercalator-Ir (Fluidigm) used at 1:2000. Cells were measured using the cyTOF 2 (Fluidigm) and injected using the supersampler. EU beads (Fluidigm) were added just before runtime (1:10 dilution) to normalize signal over runtime.

Quality Control and Data Cleaning

Normalization over run time was performed using the EU beads using the previously published bead normalized (v0.3) available here: https://github.com/nolanlab/bead-normalization/releases with the default parameters. Samples were then debarcoded using the single-cell debarcader available here: https://github.com/nolanlab/single-cell-debarcoder using the default parameters. Channel values were arcsine transformed and normalized between the two independent runs using two OA patients that were loaded in both runs. The tower independent runs were normalized to each other. Next, we selected for live cells by gating for cisplatin negative, DNA (Ir195) positive cells. Finally, from live cells, we gated for SOX9/CD44 double positive cells, which were included in the final analysis. On average, 98% of the OA and normal cells were live, and 95% and 64% respectively, were in the SOX9/CD44 gate. Gating was performed using cytobank.

FlowSOM Analysis and tSNE Projections

Clusters were called using FlowSOM (See for example Ref. 10). Analysis was performed using cytobank online implementation using the standard settings. Clustering was performed using the cell surface receptors, HIF2A and SOD2—no signaling markers were included. The self-organizing map (SOM) was constructed using the 20 OA and 5 normal samples, and then same SOM was applied to the treated samples. tSNE projection was also performed using Cytobank's online platform. All results, including flowSOM clusters and tSNE coordinates were exported as text files and manipulated for plotting in python. The results from flowSOM clusters was compared to other clustering algorithms, including SPADE and X-shift and obtained similar numbers of clusters and patterns of expression within each cluster.

Data Visualization

Data was visualized using python and the numpy (https://www.numpy.org/), pandas (https://pandas.pydata.org/pandas-docs/stable/) and seaborn (https://seaborn.pydata.org/) packages.

Reanalysis of Single Cell RNA-Sequencing Data from GSE104782

Gene counts were downloaded from GEO and reanalyzed using custom python scripts. Gene expression networks and pathway analysis were performed using IPA (Qiagen), Enrichr and STRING.

Statistical Analysis

Planned comparisons were performed with the GraphPad software Prism. The following were used: (1) one-way ANOVA followed by Tukey's post hoc test to identify specific differences between drug treatment groups, or between selected OA patient groups. For treatments, groups were only compared against DMSO controls, not against each other; (2) non-parametric, two-tailed Welch's t test for comparisons between only two groups. P-values were corrected for multiple hypothesis testing, such that the familywise error was capped at 0.05, using the Bonferroni correction method. The exact method and specific p values for significant comparisons are stated in the appropriate results section. For cyTOF plots, although only 9000 cells were visualized on the tSNE plots in the figures, average values and other calculations or statistics were performed with all cells that met the required criteria.

### Example 4: In Vivo Inhibition of Inf-A Populations Shows Therapeutic Effects in a Mouse Model of OA

Using a mouse model of post-traumatic osteoarthritis (PTOA) wherein mechanical loading can lead to onset and progression of OA (Christiansen et al. 2012), Applicant tested if early intervention with small molecule modulators of the Inf A populations can modulate the onset or progression of OA in vivo. For these experiments, wildtype C57BL mice were injected with (a) control or (b) Inf-A inhibitor (JNK II inhibitor) OA initiation to test whether these putative modulations can be therapeutic in OA. The injections were given starting 1 week after tibial loading and at a frequency of 2 injections every week to ensure their efficacy. For histological assessment, the OARSI scoring criteria was utilized to grade the OA severity with the score of 0-6 grades ranging from intact cartilage to erosion and deformation of cartilage/bone. For the quantification of OA development in each control or injured joint, 6 sagittal sections (4 μm thickness) spaced 50 μm are typically cut and stained in order to cover the whole joint compartment. A maximum score for the overall section (both tibial plateau and femoral condyle) is obtained after assigning the most severe damage score for each section and averaging the scores; a summit score (sum of the calculated scores) is calculated as a measure of the prevalent damage over the whole joint.

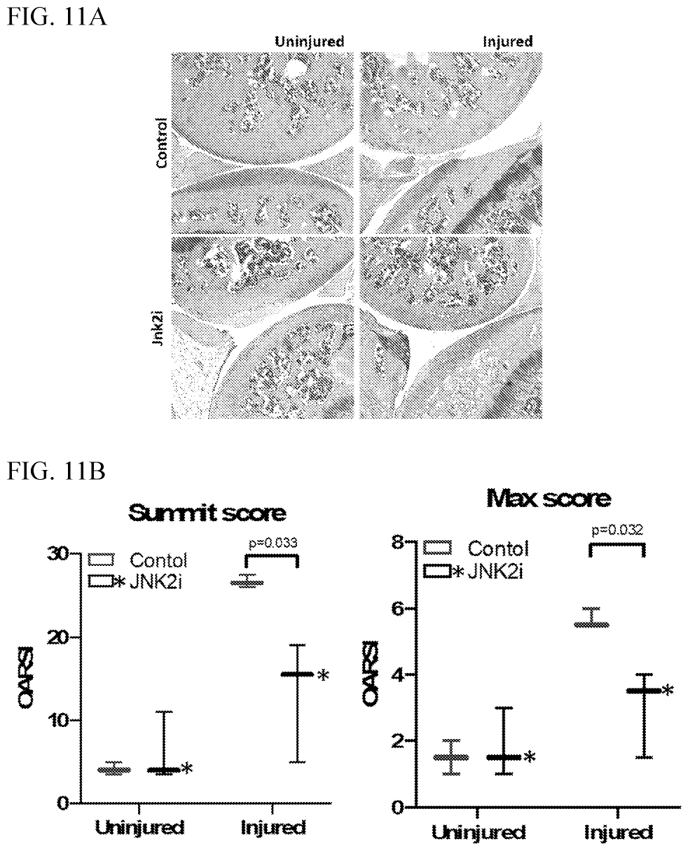

Results show that using 50 uM JNK II inhibitor with the regime described above slowed OA progression at 8 weeks post-injury compared to control as shown by reduced summit and max scores (FIGS. 11A and 11B).

### REFERENCES

1. Jiang, Y. & Tuan, R. S. Origin and function of cartilage stem/progenitor cells in osteoarthritis. Nat. Rev. Rheumatol. 11, 206-212 (2015).
2. Fernandes, A. M. Similar properties of chondrocytes from osteoarthritis joints and mesenchymal stem cells from healthy donors for tissue engineering of articular cartilage. P Lo One 8, e62994 (2013).
3. Koelling, S. Migratory chondrogenic progenitor cells from repair tissue during the later stages of human osteoarthritis. Cell Stem Cell 4, 324-335 (2009).
4. Fickert, S., Fiedler, J. & Brenner, R. E. Identification of subpopulations with characteristics of mesenchymal progenitor cells from human osteoarthritic cartilage using triple staining for cell surface markers. Arthritis Res Ther 6, R422-R432 (2004).
5. Chan, C. K. F. et al. Identification of the Human Skeletal Stem Cell. Cell 175, 43-56.e21 (2018).
6. Mathiessen, A. & Conaghan, P. G. Synovitis in osteoarthritis: current understanding with therapeutic implications. Arthritis Res. Ther. 19, (2017).
7. Mabey, T. & Honsawek, S. Cytokines as biochemical markers for knee osteoarthritis. World J. Orthop. 6, 95-105 (2015).
8. Bendall, S. C. et al. Single-Cell Mass Cytometry of Differential Immune and Drug Responses Across a Human Hematopoietic Continuum. Science 332, 687-696 (2011).
9. Taylor, S. E. B., Smeriglio, P., Dhulipala, L., Rath, M. & Bhutani, N. A global increase in 5-hydroxymethylcytosine levels marks osteoarthritic chondrocytes. Arthritis Rheumatol. Hoboken N.J. 66, 90-100 (2014).
10. Van Gassen, S. et al. FlowSOM: Using self-organizing maps for visualization and interpretation of cytometry data. Cytom. Part J. Int. Soc. Anal. Cytol. 87, 636-645 (2015).
11. Alsalameh, S., Amin, R., Gemba, T. & Lotz, M. Identification of mesenchymal progenitor cells in normal and osteoarthritic human articular cartilage. Arthritis Rheum 50, 1522-1532 (2004).
12. Bernstein, P., Sperling, I., Corbeil, D., Hempel, U. & Fickert, S. Progenitor cells from cartilage-no osteoarthritis-grade-specific differences in stem cell marker expression. Biotechnol Prog 29, 206-212 (2013).
13. Dowthwaite, G. P. et al. The surface of articular cartilage contains a progenitor cell population. J. Cell Sci. 117, 889-897 (2004).
14. Grogan, S. P., Miyaki, S., Asahara, H., D'Lima, D. D. & Lotz, M. K. Mesenchymal progenitor cell markers in human articular cartilage: normal distribution and changes in osteoarthritis. Arthritis Res. Ther. 11, R85 (2009).
15. Hattori, S., Oxford, C. & Reddi, A. H. Identification of superficial zone articular chondrocyte stem/progenitor cells. Biochem Biophys Res Commun 358, 99-103 (2007).
16. Jiang, Y. et al. Cartilage stem/progenitor cells are activated in osteoarthritis via interleukin-1β/nerve growth factor signaling. Arthritis Res. Ther. 17, (2015).
17. Hiraoka, K., Grogan, S., Olee, T. & Lotz, M. Mesenchymal progenitor cells in adult human articular cartilage. Biorheology 43, 447-454 (2006).
18. McCarthy, H. E., Bara, J. J., Brakspear, K., Singhrao, S. K. & Archer, C. W. The comparison of equine articular cartilage progenitor cells and bone marrow-derived stromal cells as potential cell sources for cartilage repair in the horse. Vet J 192, 345-351 (2012).
19. Barbero, A., Ploegert, S., Heberer, M. & Martin, I. Plasticity of clonal populations of dedifferentiated adult human articular chondrocytes. Arthritis Rheum 48, 1315-1325 (2003).
20. Williams, R. Identification and clonal characterisation of a progenitor cell sub-population in normal human articular cartilage. P Lo One 5, e13246 (2010).
21. Unguryte, A. et al. Human articular chondrocytes with higher aldehyde dehydrogenase activity have stronger expression of COL2A1 and SOX9. Osteoarthritis Cartilage 24, 873-882 (2016).
22. Grogan, S. P. et al. Identification of markers to characterize and sort human articular chondrocytes with enhanced in vitro chondrogenic capacity. Arthritis Rheum. 56, 586-595 (2007).
23. Karlsson, C. & Lindahl, A. Articular cartilage stem cell signalling. Arthritis Res Ther 11, 121 (2009).
24. Mohan, G. et al. Kartogenin treatment prevented joint degeneration in a rodent model of osteoarthritis: A pilot study. J. Orthop. Res. 34, 1780-1789 (2016).
25. Kwon, J. Y. et al. Kartogenin inhibits pain behavior, chondrocyte inflammation, and attenuates osteoarthritis progression in mice through induction of IL-10. Sci. Rep. 8, 13832 (2018).
26. Johnson, K. et al. A Stem Cell-Based Approach to Cartilage Repair. Science 336, 717-721 (2012).
27. Ji, Q. et al. Single-cell RNA-seq analysis reveals the progression of human osteoarthritis. Ann. Rheum. Dis. annrheumdis-2017-212863 (2018). doi:10.1136/annrheumdis-2017-212863
28. Longobardi, L. et al. Associations between the chemokine biomarker CCL2 and knee osteoarthritis outcomes: the Johnston County Osteoarthritis Project. Osteoarthritis Cartilage 26, 1257-1261 (2018).
29. Raghu, H. et al. CCL2/CCR2, but not CCL5/CCR5, mediates monocyte recruitment, inflammation and cartilage destruction in osteoarthritis. Ann. Rheum. Dis. 76, 914-922 (2017).
30. Burke, J. R. et al. BMS-345541 is a highly selective inhibitor of I kappa B kinase that binds at an allosteric site of the enzyme and blocks NF-kappa B-dependent transcription in mice. J Biol. Chem. 278, 1450-1456 (2003).
31. van Caam, A. et al. TGFβ1-induced SMAD2/3 and SMAD1/5 phosphorylation are both ALK5-kinase-dependent in primary chondrocytes and mediated by TAK1 kinase activity. Arthritis Res. Ther. 19, 112 (2017).
32. Lee, J., Smeriglio, P., Dragoo, J., Maloney, W. J. & Bhutani, N. CD24 enrichment protects while its loss increases susceptibility of juvenile chondrocytes towards inflammation. Arthritis Res. Ther. 18, (2016).
33. Smith, N. C., Fairbridge, N. A., Pallegar, N. K. & Christian, S. L. Dynamic upregulation of CD24 in pre-adipocytes promotes adipogenesis. Adipocyte 4, 89-100 (2015).
34. Jeon, O. H. et al. Local clearance of senescent cells attenuates the development of post-traumatic osteoarthritis and creates a pro-regenerative environment. Nat. Med. 23, 775-781 (2017).
35. Deshmane, S. L., Kremlev, S., Amini, S. & Sawaya, B. E. Monocyte Chemoattractant Protein-1 (MCP-1): An Overview. J. Interferon Cytokine Res. 29, 313-326 (2009).
36. Tsou, C.-L. et al. Critical roles for CCR2 and MCP-3 in monocyte mobilization from bone marrow and recruitment to inflammatory sites. J. Clin. Invest. 117, 902-909 (2007).
37. Han, Z. et al. c-Jun N-terminal kinase is required for metalloproteinase expression and joint destruction in inflammatory arthritis. J. Clin. Invest. 108, 73-81 (2001).
38. Schepetkin, I. A. et al. Anti-Inflammatory Effects and Joint Protection in Collagen-Induced Arthritis after Treatment with IQ-1S, a Selective c-Jun N-Terminal Kinase Inhibitor. J Pharmacol. Exp. Ther. 353, 505-516 (2015).
39. Bluml, S., Scheinecker, C., Smolen, J. S. & Redlich, K. Targeting TNF receptors in rheumatoid arthritis. Int. Immunol. 24, 275-281 (2012).
40. Soul, J. et al. Stratification of knee osteoarthritis: two major patient subgroups identified by genome-wide expression analysis of articular cartilage. Ann. Rheum. Dis. 77, 423-423 (2018).
41. Rushton, M. D. et al. Characterization of the cartilage DNA methylome in knee and hip osteoarthritis. Arthritis Rheumatol. Hoboken N.J. 66, 2450-2460 (2014).
42. Fernández-Tajes, J. et al. Genome-wide DNA methylation analysis of articular chondrocytes reveals a cluster of osteoarthritic patients. Ann. Rheum. Dis. 73, 668-677 (2014).
43. Murahashi, Y. et al. Intra-articular administration of IκBα kinase inhibitor suppresses mouse knee osteoarthritis via downregulation of the NF-κB/HIF-2α axis. Sci. Rep. 8, 16475 (2018).
44. Inman, G. J. et al. SB-431542 is a potent and specific inhibitor of transforming growth factor-beta superfamily type I activin receptor-like kinase (ALK) receptors ALK4, ALK5, and ALK7. Mol. Pharmacol. 62, 65-74 (2002).
45. Laping, N. J. et al. Inhibition of transforming growth factor (TGF)-beta1-induced extracellular matrix with a novel inhibitor of the TGF-beta type I receptor kinase activity: SB-431542. Mol. Pharmacol. 62, 58-64 (2002).

1. A method of treating osteoarthritis in a subject in need thereof comprising administering an effective amount of an activin-like kinase 5 (Alk5) inhibitor, a c-Jun N-terminal kinase (JNK) inhibitor, a tumor necrosis factor receptor II (TNFR II) inhibitor, an interleukin 1 receptor type 1 (IL1R1) inhibitor, or a CD24 activator.

2. The method of claim 1, wherein
i) the Alk5 inhibitor is an antibody, a nucleic acid, or a small molecule;
ii) the JNK inhibitor is an antibody, a nucleic acid, or a small molecule;
iii) the TNFR II inhibitor is an antibody, a nucleic acid, or a small molecule;
iv) the IL1R1 receptor inhibitor is an antibody, a nucleic acid, or a small molecule; and/or
v) the CD24 activator is an antibody, nucleic acid, or a small molecule.

3. The method of claim 1, wherein
i) the Alk5 inhibitor is selected from SB431542, Galunisertib, A 83-01, A 77-01, SB 505124, R 268712, IN 1130, SM 16, AZ 12799734, and LY 364947;
ii) the JNK inhibitor is selected from SP600125, TCS JNK6o, SU 3327, CEP 1347, c-JUN peptide, AEG 3481, TCS JNK 5a, BI 78D3, IQ3, SR 3576, IQ 1S, JIP-1, and CC401 dihydrochloride; and/or
iii) the CD24 activator is 3-Isobutyl-1-methylxanthine (IBMX).

4. The method of claim 3, wherein the Alk5 inhibitor is SB431542.

5. (canceled)

6. (canceled)

7. The method of claim 1, wherein the JNK kinase inhibitor is selected from a JNK1 inhibitor and a JNK2 inhibitor.

8. (canceled)

9. (canceled)

10. (canceled)

11. (canceled)

12. The method of claim 1, comprising administering an effective amount of
i) an activin-like kinase 5 (Alk5) inhibitor and a CD24 activator;
ii) a c-Jun N-terminal kinase (JNK) inhibitor and a CD24 activator;
iii) a tumor necrosis factor receptor II (TNFR II) inhibitor;
iv) an interleukin 1 receptor type 1 (IL1R1) inhibitor;
v) a tumor necrosis factor receptor II (TNFR II) inhibitor and a CD24 activator; and/or
vi) an interleukin 1 receptor type 1 (IL1R1) inhibitor and a CD24 activator.

13. (canceled)

14. (canceled)

15. (canceled)

16. (canceled)

17. (canceled)

18. The method of claim 1, further comprising administering a pain medication.

19. The method of claim 18, wherein the pain medication is selected from a non-steroidal anti-inflammatory drug (NSAID), a corticosteroid, a hyaluronic acid, and an opioid.

20. The method of claim 1, wherein said administering is intraarticularly administering.

21. The method of claim 12, wherein said administering is subsequent to said administering of said pain medication.

22. The method of claim 1, wherein the subject is determined to have osteoarthritis by one or more of a physical examination, an x-ray examination, arthroscopic examination, a magnetic resonance examination, and arthrocentesis.

23. The method of claim 1, wherein said treating is reducing the progression of said osteoarthritis.

24. A method of treating osteoarthritis in a patient in need thereof comprising administering to the patient an inhibitor of an inflammation amplifying (Inf-A) population of chondrocytes and an activator of an inflammation dampening (Inf-D) population of chondrocytes.

25. A composition for the treatment of osteoarthritis comprising an inhibitor of an inflammation amplifying (Inf-A) population of chondrocytes and an activator of an inflammation dampening (Inf-D) population of chondrocytes.

26. The composition of claim 25 wherein the inhibitor of an inflammation amplifying (Inf-A) population of chondrocytes is selected from an activin-like kinase 5 (Alk5) inhibitor, a c-Jun N-terminal kinase (JNK) inhibitor, a tumor necrosis factor receptor II (TNFR II) inhibitor, and an interleukin 1 receptor type 1 (IL1R1) inhibitor.

27. The composition of claim 25 wherein the activator of an inflammation dampening (Inf-D) population of chondrocytes is a CD24 activator.

28. The composition of claim 26, wherein
i) the Alk5 inhibitor is an antibody, a nucleic acid, or a small molecule;
ii) the JNK inhibitor is an antibody, a nucleic acid, or a small molecule;
iii) the TNFR II inhibitor is an antibody, a nucleic acid, or a small molecule;
iv) the IL1R1 receptor inhibitor is an antibody, a nucleic acid, or a small molecule; and/or
v) the CD24 activator is an antibody, nucleic acid, or a small molecule.

29. The composition of claim 26, wherein
i) the Alk5 inhibitor is selected from SB431542, Galunisertib, A 83-01, A 77-01, SB 505124, R 268712, IN 1130, SM 16, AZ 12799734, and LY 364947;
ii) the JNK inhibitor is selected from SP600125, TCS JNK6o, SU 3327, CEP 1347, c-JUN peptide, AEG 3481, TCS JNK 5a, BI 78D3, IQ3, SR 3576, IQ 1S, JIP-1, and CC401 dihydrochloride; and/or
iii) the CD24 activator is 3-Isobutyl-1-methylxanthine (IBMX).

30. The composition of claim 29, wherein the Alk5 inhibitor is SB431542.

31. (canceled)

32. (canceled)

33. The composition of claim 26, wherein the JNK kinase inhibitor is selected from a JNK1 inhibitor and a JNK2 inhibitor.

34. (canceled)

35. (canceled)

36. (canceled)

37. (canceled)

38. The composition of claim 25, comprising an effective amount of
i) an activin-like kinase 5 (Alk5) inhibitor and a CD24 activator;
ii) a c-Jun N-terminal kinase (JNK) inhibitor and a CD24 activator;
iii) a tumor necrosis factor receptor II (TNFR II) inhibitor;
iv) an interleukin 1 receptor type 1 (IL1R1) inhibitor;
v) a tumor necrosis factor receptor II (TNFR II) inhibitor and a CD24 activator; and/or
vi) an interleukin 1 receptor type 1 (IL1R1) inhibitor and a CD24 activator.

39. (canceled)

40. (canceled)

41. (canceled)

42. (canceled)

43. (canceled)
